SEQUENCE LISTING (120 sequences)

SEQ ID NO: 1 - protein, 6 aa, synthetic construct
SSAAIN

SEQ ID NO: 2 - protein, 17 aa, synthetic construct
YIYAGSGSTYYASWAKG

SEQ ID NO: 3 - protein, 11 aa, synthetic construct
VRSLSGGDRNL

SEQ ID NO: 4 - protein, 13 aa, synthetic construct
QSSESVSKNDYLA

SEQ ID NO: 5 - protein, 10 aa, synthetic construct
LLIYRASTLA

SEQ ID NO: 6 - protein, 11 aa, synthetic construct
QGGYSRNNDMT

SEQ ID NO: 7 - protein, 6 aa, synthetic construct
SSAAIN

SEQ ID NO: 8 - protein, 17 aa, synthetic construct
YIYAGDDSIYYASWAKG

SEQ ID NO: 9 - protein, 11 aa, synthetic construct
ARSLSGGDRNL

SEQ ID NO: 10 - protein, 13 aa, synthetic construct
QSSESVSKNNYLA

SEQ ID NO: 11 - protein, 10 aa, synthetic construct
LLIYEASKLP

SEQ ID NO: 12 - protein, 11 aa, synthetic construct
QGGYSRNNDMT

SEQ ID NO: 13 - protein, 6 aa, synthetic construct
SSAAIN

SEQ ID NO: 14 - protein, 17 aa, synthetic construct
YIYAGSASTYYATWAKG

SEQ ID NO: 15 - protein, 11 aa, synthetic construct
ARSLSGGDRNL

SEQ ID NO: 16 - protein, 13 aa, synthetic construct
QSSESVTKNNYLA

SEQ ID NO: 17 - protein, 10 aa, synthetic construct
LLIYEASKLA

SEQ ID NO: 18 - protein, 11 aa, synthetic construct
QGGYSRNNDMT

SEQ ID NO: 19 - protein, 6 aa, synthetic construct
SSAAIN

SEQ ID NO: 20 - protein, 17 aa, synthetic construct
YIYAGSGSTYYASWAKG

SEQ ID NO: 21 - protein, 11 aa, synthetic construct
ARSLSGGDRNL

SEQ ID NO: 22 - protein, 13 aa, synthetic construct
QSSENVAKNNYLA

SEQ ID NO: 23 - protein, 10 aa, synthetic construct
LLIYEASKLA

SEQ ID NO: 24 - protein, 11 aa, synthetic construct
QGGYNRNNDMT

SEQ ID NO: 25 - protein, 6 aa, synthetic construct
SSAAIN

SEQ ID NO: 26 - protein, 17 aa, synthetic construct
YIYAGSGSTYYASWAKG

SEQ ID NO: 27 - protein, 11 aa, synthetic construct
ARSLSGGDRNL

SEQ ID NO: 28 - protein, 13 aa, synthetic construct
QSSESVSKNNYLA

SEQ ID NO: 29 - protein, 10 aa, synthetic construct
LLIYEASKLA

SEQ ID NO: 30 - protein, 11 aa, synthetic construct
QGGYSRNNDMT

SEQ ID NO: 31 - protein, 6 aa, synthetic construct
SKYGVN

SEQ ID NO: 32 - protein, 16 aa, synthetic construct
WITSDGITFYASWAKG

SEQ ID NO: 33 - protein, 11 aa, synthetic construct
TRSILSAWDNI

SEQ ID NO: 34 - protein, 13 aa, synthetic construct
QSSKSVYNGNWLS

SEQ ID NO: 35 - protein, 10 aa, synthetic construct
RLIYSASTLA

SEQ ID NO: 36 - protein, 10 aa, synthetic construct
AGGYSGEIDD

SEQ ID NO: 37 - protein, 6 aa, synthetic construct
SSYYVS

SEQ ID NO: 38 - protein, 16 aa, synthetic construct
GINTGGVAYYATWAKG

SEQ ID NO: 39 - protein, 12 aa, synthetic construct
ARHPGGDAGTDL

SEQ ID NO: 40 - protein, 11 aa, synthetic construct
QASQSITKYLA

SEQ ID NO: 41 - protein, 10 aa, synthetic construct
RLIYKTSTLA

SEQ ID NO: 42 - protein, 14 aa, synthetic construct
QNNVPGKITDSYGA

SEQ ID NO: 43 - protein, 8 aa, synthetic construct
STFGMGVS

SEQ ID NO: 44 - protein, 16 aa, synthetic construct
HIYWDDVKRYNPSLKS

SEQ ID NO: 45 - protein, 10 aa, synthetic construct
ARTAGAWFAF

SEQ ID NO: 46 - protein, 10 aa, synthetic construct
SARSTVNYMH

SEQ ID NO: 47 - protein, 7 aa, synthetic construct
DTSKLAS

SEQ ID NO: 48 - protein, 9 aa, synthetic construct
QQWTSNLLT

SEQ ID NO: 49 - protein, 8 aa, synthetic construct
STYGMGIG

SEQ ID NO: 50 - protein, 16 aa, synthetic construct
HIWWDDVKRYDPVLKS

SEQ ID NO: 51 - protein, 12 aa, synthetic construct
ARIPGDGSHSDY

SEQ ID NO: 52 - protein, 16 aa, synthetic construct
RSTQSLVHSNGITYLE

SEQ ID NO: 53 - protein, 7 aa, synthetic construct
RISNRFS

SEQ ID NO: 54 - protein, 9 aa, synthetic construct
FQGSHGPYT

SEQ ID NO: 55 - protein, 8 aa, synthetic construct
STSGMGIG

SEQ ID NO: 56 - protein, 16 aa, synthetic construct
HIWWDDVKRYDPALKS

SEQ ID NO: 57 - protein, 12 aa, synthetic construct
ARIPGDGSHFDY

SEQ ID NO: 58 - protein, 16 aa, synthetic construct
RSSQSLVHSNGITYLE

SEQ ID NO: 59 - protein, 7 aa, synthetic construct
RVSNRFS

SEQ ID NO: 60 - protein, 9 aa, synthetic construct
FQGSHGPYT

SEQ ID NO: 61 - DNA, 18 bp, synthetic construct
  source 1..18: mol_type = other DNA
agtagcgctgcaataaac

SEQ ID NO: 62 - DNA, 51 bp, synthetic construct
  source 1..51: mol_type = other DNA
tacatttatgctggtagtggtagcacatactacgcgagctgggcgaaaggc

SEQ ID NO: 63 - DNA, 33 bp, synthetic construct
  source 1..33: mol_type = other DNA
gtcagaagtttaagtggtggtgatcggaacttg

SEQ ID NO: 64 - DNA, 39 bp, synthetic construct
  source 1..39: mol_type = other DNA
cagtccagtgagagtgtttctaagaatgattacttagcc

SEQ ID NO: 65 - DNA, 30 bp, synthetic construct
  source 1..30: mol_type = other DNA
ctcctgatctacagggcttccactctggca

SEQ ID NO: 66 - DNA, 33 bp, synthetic construct
  source 1..33: mol_type = other DNA
caaggcggttatagtaggaataatgatatgact

SEQ ID NO: 67 - DNA, 18 bp, synthetic construct
  source 1..18: mol_type = other DNA
agtagcgctgcaataaac

SEQ ID NO: 68 - DNA, 51 bp, synthetic construct
  source 1..51: mol_type = other DNA
tacatttatgctggtgatgatagtatatactacgcgagctgggcgaaaggc

SEQ ID NO: 69 - DNA, 33 bp, synthetic construct
  source 1..33: mol_type = other DNA
gccagaagtttgagtggtggtgatcggaacttg

SEQ ID NO: 70 - DNA, 39 bp, synthetic construct
  source 1..39: mol_type = other DNA
cagtccagtgagagtgtttctaagaacaactacttagcc

SEQ ID NO: 71 - DNA, 30 bp, synthetic construct
  source 1..30: mol_type = other DNA
ctcctgatctacgaagcatccaaactgcca

SEQ ID NO: 72 - DNA, 33 bp, synthetic construct; identical to SEQ ID NO: 66
  source 1..33: mol_type = other DNA

SEQ ID NO: 73 - DNA, 18 bp, synthetic construct
  source 1..18: mol_type = other DNA
agtagcgctgcaataaac

SEQ ID NO: 74 - DNA, 51 bp, synthetic construct
  source 1..51: mol_type = other DNA
tacatttatgctggtagtgctagcacatactacgcgacctgggcgaaaggc

SEQ ID NO: 75 - DNA, 33 bp, synthetic construct; identical to SEQ ID NO: 69
  source 1..33: mol_type = other DNA

SEQ ID NO: 76 - DNA, 39 bp, synthetic construct
  source 1..39: mol_type = other DNA
cagtccagtgagagtgttactaagaacaactacttagcc

SEQ ID NO: 77 - DNA, 30 bp, synthetic construct
  source 1..30: mol_type = other DNA
ctcctgatctacgaagcatccaaactggca

SEQ ID NO: 78 - DNA, 33 bp, synthetic construct; identical to SEQ ID NO: 66
  source 1..33: mol_type = other DNA

SEQ ID NO: 79 - DNA, 18 bp, synthetic construct
  source 1..18: mol_type = other DNA
agtagcgctgcaataaat

SEQ ID NO: 80 - DNA, 51 bp, synthetic construct; identical to SEQ ID NO: 62
  source 1..51: mol_type = other DNA

SEQ ID NO: 81 - DNA, 33 bp, synthetic construct
  source 1..33: mol_type = other DNA
gccagaagtttgagtggcggtgatcggaacttg

SEQ ID NO: 82 - DNA, 39 bp, synthetic construct
  source 1..39: mol_type = other DNA
cagtccagtgagaatgttgctaagaacaactacttagcc

SEQ ID NO: 83 - DNA, 30 bp, synthetic construct; identical to SEQ ID NO: 77
  source 1..30: mol_type = other DNA

SEQ ID NO: 84 - DNA, 33 bp, synthetic construct
  source 1..33: mol_type = other DNA
caaggcggttataataggaataatgatatgact

SEQ ID NO: 85 - DNA, 18 bp, synthetic construct
  source 1..18: mol_type = other DNA
agtagcgctgcaataaat

SEQ ID NO: 86 - DNA, 51 bp, synthetic construct; identical to SEQ ID NO: 62
  source 1..51: mol_type = other DNA

SEQ ID NO: 87 - DNA, 33 bp, synthetic construct; identical to SEQ ID NO: 81
  source 1..33: mol_type = other DNA

SEQ ID NO: 88 - DNA, 39 bp, synthetic construct; identical to SEQ ID NO: 70
  source 1..39: mol_type = other DNA

SEQ ID NO: 89 - DNA, 30 bp, synthetic construct; identical to SEQ ID NO: 77
  source 1..30: mol_type = other DNA

SEQ ID NO: 90 - DNA, 33 bp, synthetic construct; identical to SEQ ID NO: 66
  source 1..33: mol_type = other DNA

SEQ ID NO: 91 - DNA, 18 bp, synthetic construct
  source 1..18: mol_type = other DNA
agtaaatatggagtgaac

SEQ ID NO: 92 - DNA, 48 bp, synthetic construct
  source 1..48: mol_type = other DNA
tggattactagtgatggtatcacattctacgcgagctgggcgaaaggc

SEQ ID NO: 93 - DNA, 33 bp, synthetic construct
  source 1..33: mol_type = other DNA
accagaagtatacttagtgcttgggataacatc

SEQ ID NO: 94 - DNA, 39 bp, synthetic construct
  source 1..39: mol_type = other DNA
cagtccagtaagagtgtttataatgggaactggttatcc

SEQ ID NO: 95 - DNA, 30 bp, synthetic construct
  source 1..30: mol_type = other DNA
cgcctgatctattctgcatccactctggca

SEQ ID NO: 96 - DNA, 30 bp, synthetic construct
  source 1..30: mol_type = other DNA
gcaggcggttatagtggtgagattgatgat

SEQ ID NO: 97 - DNA, 18 bp, synthetic construct
  source 1..18: mol_type = other DNA
agtagctactacgtgagc

SEQ ID NO: 98 - DNA, 48 bp, synthetic construct
  source 1..48: mol_type = other DNA
ggcattaatactggtggtgtcgcatactacgcgacctgggcaaaaggc

SEQ ID NO: 99 - DNA, 36 bp, synthetic construct
  source 1..36: mol_type = other DNA
gccagacatcctggtggtgatgctggtaccgacttg

SEQ ID NO: 100 - DNA, 33 bp, synthetic construct
  source 1..33: mol_type = other DNA
caggccagtcagagcattactaaatacttagcc

SEQ ID NO: 101 - DNA, 30 bp, synthetic construct
  source 1..30: mol_type = other DNA
cgcctgatctacaagacatccactctggca

SEQ ID NO: 102 - DNA, 42 bp, synthetic construct
  source 1..42: mol_type = other DNA
caaaacaatgttcctggtaaaattactgacagttatggggct

SEQ ID NO: 103 - DNA, 24 bp, synthetic construct
  source 1..24: mol_type = other DNA
agcacttttggtatgggtgtgagc

SEQ ID NO: 104 - DNA, 48 bp, synthetic construct
  source 1..48: mol_type = other DNA
cacatttactgggatgatgtcaagcgctataatccatccctgaagagc

SEQ ID NO: 105 - DNA, 30 bp, synthetic construct
  source 1..30: mol_type = other DNA
gctcgaaccgctggggcctggtttgctttc

SEQ ID NO: 106 - DNA, 30 bp, synthetic construct
  source 1..30: mol_type = other DNA
agtgccaggtcaactgtaaattacatgcac

SEQ ID NO: 107 - DNA, 21 bp, synthetic construct
  source 1..21: mol_type = other DNA
gacacatccaaactggcttct

SEQ ID NO: 108 - DNA, 27 bp, synthetic construct
  source 1..27: mol_type = other DNA
cagcagtggactagtaatttgctcacg

SEQ ID NO: 109 - DNA, 24 bp, synthetic construct
  source 1..24: mol_type = other DNA
agcacttatggtatgggtataggc

SEQ ID NO: 110 - DNA, 48 bp, synthetic construct
  source 1..48: mol_type = other DNA
cacatttggtgggatgatgtcaagcgctatgatccagtcctgaagagc

SEQ ID NO: 111 - DNA, 36 bp, synthetic construct
  source 1..36: mol_type = other DNA
gctcgcatacctggggatggttcccacagtgactac

SEQ ID NO: 112 - DNA, 48 bp, synthetic construct
  source 1..48: mol_type = other DNA
agatctactcagagccttgtacatagtaatggaatcacctatttagag

SEQ ID NO: 113 - DNA, 21 bp, synthetic construct
  source 1..21: mol_type = other DNA
agaatttccaaccgattttct

SEQ ID NO: 114 - DNA, 27 bp, synthetic construct
  source 1..27: mol_type = other DNA
tttcaaggttcacatggtccgtacacg

SEQ ID NO: 115 - DNA, 24 bp, synthetic construct
  source 1..24: mol_type = other DNA
agcacttctggtatgggtataggc

SEQ ID NO: 116 - DNA, 48 bp, synthetic construct
  source 1..48: mol_type = other DNA
cacatttggtgggatgatgtcaagcgctatgatccagccctgaagagc

SEQ ID NO: 117 - DNA, 36 bp, synthetic construct
  source 1..36: mol_type = other DNA
gctcgaatacctggggatggttcccactttgactac

SEQ ID NO: 118 - DNA, 48 bp, synthetic construct
  source 1..48: mol_type = other DNA
agatctagtcagagccttgtacatagtaatggaatcacctatttagag

SEQ ID NO: 119 - DNA, 21 bp, synthetic construct
  source 1..21: mol_type = other DNA
agagtttccaaccgattttct

SEQ ID NO: 120 - DNA, 27 bp, synthetic construct
  source 1..27: mol_type = other DNA
tttcaaggttcacatggtccgtacacg

(19) United States
(12) Patent Application Publication
(10) Pub. No.: US 2026/0159576 A1
(43) Pub. Date: Jun. 11, 2026

(54) ANTIBODY OR ANTIGEN-BINDING FRAGMENT THEREOF BINDING TO ß-CTx AND KIT THEREOF

(71) Applicants: Shenzhen Mindray Bio-Medical Electronics Co., Ltd., Shenzhen (CN); HyTest Ltd., Turku (FI)

(72) Inventors: Alexander B. Postnikov, Moscow (RU); Tatiana A. Molchanova, Moscow (RU); Anastasia V. Bereznikova, Moscow (RU); Anna V. Rusanova, Moscow (RU); Elena A. Dergousova, Moscow (RU); Stanislav V. Kozlovsky, Moscow (RU); Natalia A. Gruzdeva, Moscow (RU); Anna A. Trufanova, Moscow (RU); Alexander S. Mishin, Moscow (RU); Fedor N. Rozov, Moscow (RU); Sergei A. Kotlov, Moscow (RU); Alexey G. Katrukha, Moscow (RU); Shibo Zhu, Shenzhen (CN); Chengxiong Zhan, Shenzhen (CN); Tao Tang, Shenzhen (CN); Lingjia Wu, Shenzhen (CN)

(73) Assignees: Shenzhen Mindray Bio-Medical Electronics Co., Ltd., Shenzhen (CN); HyTest Ltd., Turku (FI)

(21) Appl. No.: 19/258,929

(22) Filed: Jul. 3, 2025

(30) Foreign Application Priority Data
Jul. 3, 2024 (CN) 202410890870.4

(51) Int. Cl. C07K 16/18; G01N 33/68

(52) U.S. Cl. CPC C07K 16/18; G01N 33/6893; C07K 2317/33; C07K 2317/34; C07K 2317/565; C07K 2317/92; G01N 2333/78; G01N 2800/108

(57) ABSTRACT

The present application relates to an antibody or an antigen-binding fragment thereof binding to β-CTx. The antibody or an antigen-binding fragment thereof comprises a heavy chain variable region and a light chain variable region. The heavy chain variable region and the light chain variable region comprise HCDR and LCDR sequences of the present application. The present application also relates to a corresponding isolated nucleic acid molecule, expression vector and host cell. In addition, the application also relates to a kit and a method for diagnosing osteoporosis in a subject.

### CROSS REFERENCE

This application claims benefit of priority of Chinese Patent Application No. 202410890870.4 filed on Jul. 3, 2024, the content of which is incorporated herein by reference in its entirety.

### SEQUENCE LISTING

This application incorporates by reference a Sequence Listing submitted with this application as XML file format, entitled “P2024119US1_sequence listing.xml” created on Jun. 28, 2025 having a size of 106,557 bytes.

### TECHNICAL FIELD

The present application relates to the field of diagnosis of osteoporosis and, in particular, to an antibody or antigen-binding fragment thereof binding to β-CTx.

### BACKGROUND

Bone tissue is constantly remodeled through the process of bone resorption and new bone replacement. Osteoclasts are responsible for bone resorption, while osteoblasts are responsible for new bone formation.

In young people, the processes of bone resorption and bone formation remain balanced, but during menopause this balance is disturbed by increased bone loss due to increased absorption. By binding to estrogen receptors on osteoblasts, estrogen directly stimulates the proliferation of osteoblasts and inhibits the differentiation of osteoclasts. In addition, estrogen can also increase regulatory factors produced by osteoblasts, thereby preventing the activity of osteoclasts and further inhibiting bone resorption during menopause (Chubb et al., 2012, Clinical Biochemistry, 45:12, 928-935).

Insufficient calcium intake and low 25-hydroxyvitamin D (25(OH)D) levels may also accelerate age-related bone loss. The reduction in the production of the main precursor of vitamin D in the skin and the synthesis of 1,25(OH)2D (the active metabolite of vitamin D) results in reduced absorption of calcium by the kidneys and this leads to increased secretion of parathyroid hormone (PTH), thus in turn increasing bone absorption.

In the process of synthesizing the main matrix protein Type I collagen, polypeptide chains α1 and α2 are first formed. α1 and α2 combine together in a ratio of 2:1 to form a procollagen molecule having a triple helix structure. Procollagen is secreted into the extracellular environment, in which terminal peptides are isolated from the procollagen, and the resulting immature collagen is incorporated into the fibrous structure. A series of modifications of collagen molecules in the fiber components and their cross-linking with pyridinoline yield mature collagen.

The main degradation product of the Type I collagen C-terminal peptide is a structure composed of two octapeptides (8 amino acid residues, EKAH(β-D)GGR) cross-linked by pyridinoline. In newly formed bones, the octapeptide sequence contains α-aspartic acid, but as the bone ages, this amino acid isomerizes into a β-form (that is, the degree of isomerization is in direct proportion to the age of the bone). Degradation products of Type I collagen, such as the cross-linked Type I collagen carboxy-terminal telopeptide (β-CTx), have been shown to be reliable biomarkers of bone resorption and osteoporosis (Cloos et al., Biochemistry Journal, 2000, 345, 473-480).

Although methods have been developed to measure β-CTx in human serum, due to the short length of the peptide in β-CTx, this has brought great challenges to the screening of anti-β-CTx antibodies, which in turn leads to the limited number of antibodies that specifically bind to β-CTx and further limits the development of β-CTx immunoassays.

In view of this, it is desired to provide a novel antibody specifically binding to β-CTx.

### SUMMARY

In a first aspect, the present application provides an antibody or an antigen-binding fragment thereof binding to β-CTx, comprising:
a heavy chain variable region (VH) and a light chain variable region (VL),
wherein the heavy chain variable region comprises HCDR1 including an amino acid sequence as set forth in any one of SEQ ID NO: 1, 7, 13, 19, 25, 31, 37, 43, 49 or 55 or a variant thereof, HCDR2 including an amino acid sequence as set forth in any one of SEQ ID NO: 2, 8, 14, 20, 26, 32, 38, 44, 50 or 56 or a variant thereof, and HCDR3 including an amino acid sequence as set forth in any one of SEQ ID NO: 3, 9, 15, 21, 27, 33, 39, 45, 51 or 57 or a variant thereof,
wherein, the light chain variable region comprises LCDR1 including an amino acid sequence as set forth in any one of SEQ ID NO: 4, 10, 16, 22, 28, 34, 40, 46, 52 or 58 or a variant thereof, LCDR2 or including an amino acid sequence as set forth in any one of SEQ ID NO: 5, 11, 17, 23, 29, 35, 41, 47, 53 or 59 or a variant thereof, and LCDR3 including an amino acid sequence as set forth in any one of SEQ ID NO: 6, 12, 18, 24, 30, 36, 42, 48, 54 or 60 or a variant thereof, and
wherein each variant comprises one or more amino acid mutations compared to an amino acid sequence from which it is derived, and each amino acid mutation is selected from amino acid substitution, deletion or addition; preferably, the substitution is a conservative substitution; preferably, each variant has at least 80%, at least 90%, or at least 95% identity to the amino acid sequence from which it is derived.

By providing antibodies with specific HCDRs and LCDRs, the present application provides antibodies that specifically bind to β-CTx with excellent binding ability, thereby solving the problem of scarce antibody options for immunoassays of β-CTx to some extent.

In some embodiments, the antibody of the present application comprises:
HCDR1, HCDR2, HCDR3, LCDR1, LCDR2 and LCDR3 including sequences that have at least 80%, at least 90%, or at least 95% identity to SEQ ID NOs: 1-6, respectively;
HCDR1, HCDR2, HCDR3, LCDR1, LCDR2 and LCDR3 including sequences that have at least 80%, at least 90%, or at least 95% identity to SEQ ID NOs: 7-12, respectively;
HCDR1, HCDR2, HCDR3, LCDR1, LCDR2 and LCDR3 including sequences that have at least 80%, at least 90%, or at least 95% identity to SEQ ID NOs: 13-18, respectively;
HCDR1, HCDR2, HCDR3, LCDR1, LCDR2 and LCDR3 including sequences that have at least 80%, at least 90%, or at least 95% identity to SEQ ID NOs: 19-24, respectively;
HCDR1, HCDR2, HCDR3, LCDR1, LCDR2 and LCDR3 including sequences that have at least 80%, at least 90%, or at least 95% identity to SEQ ID NOs: 25-30, respectively;
HCDR1, HCDR2, HCDR3, LCDR1, LCDR2 and LCDR3 including sequences that have at least 80%, at least 90%, or at least 95% identity to SEQ ID NOs: 31-36, respectively;
HCDR1, HCDR2, HCDR3, LCDR1, LCDR2 and LCDR3 including sequences that have at least 80%, at least 90%, or at least 95% identity to SEQ ID NOs: 37-42, respectively;
HCDR1, HCDR2, HCDR3, LCDR1, LCDR2 and LCDR3 including sequences that have at least 80%, at least 90%, or at least 95% identity to SEQ ID NOs: 43-48, respectively;
HCDR1, HCDR2, HCDR3, LCDR1, LCDR2 and LCDR3 including sequences that have at least 80%, at least 90%, or at least 95% identity to SEQ ID NOs: 49-54, respectively; or
HCDR1, HCDR2, HCDR3, LCDR1, LCDR2 and LCDR3 including sequences that have at least 80%, at least 90%, or at least 95% identity to SEQ ID NOs: 55-60, respectively.

In some embodiments, the antibody of the present application comprises:
HCDR1, HCDR2, HCDR3, LCDR1, LCDR2 and LCDR3 including sequences as set forth in SEQ ID NOs: 1-6, respectively;
HCDR1, HCDR2, HCDR3, LCDR1, LCDR2 and LCDR3 including sequences as set forth in SEQ ID NOs: 7-12, respectively;
HCDR1, HCDR2, HCDR3, LCDR1, LCDR2 and LCDR3 including sequences as set forth in SEQ ID NOs: 13-18, respectively;
HCDR1, HCDR2, HCDR3, LCDR1, LCDR2 and LCDR3 including sequences as set forth in SEQ ID NOs: 19-24, respectively;
HCDR1, HCDR2, HCDR3, LCDR1, LCDR2 and LCDR3 including sequences as set forth in SEQ ID NOs: 25-30, respectively;
HCDR1, HCDR2, HCDR3, LCDR1, LCDR2 and LCDR3 including sequences as set forth in SEQ ID NOs: 31-36, respectively;
HCDR1, HCDR2, HCDR3, LCDR1, LCDR2 and LCDR3 including sequences as set forth in SEQ ID NOs: 37-42, respectively;
HCDR1, HCDR2, HCDR3, LCDR1, LCDR2 and LCDR3 including sequences as set forth in SEQ ID NOs: 43-48, respectively;
HCDR1, HCDR2, HCDR3, LCDR1, LCDR2 and LCDR3 including sequences as set forth in SEQ ID NOs: 49-54, respectively; or
HCDR1, HCDR2, HCDR3, LCDR1, LCDR2 and LCDR3 including sequences as set forth in SEQ ID NOs: 55-60, respectively.

In a specific embodiment, an antigen-binding epitope of the antibody or the antigen-binding fragment thereof of the present application is selected from one or more of the group consisting of: AH(betaD)G, AH(betaD)GG, AH(betaD)GGR and AH(betaD)GGR-COOH.

In a second aspect, the present application provides an antibody or an antigen-binding fragment thereof. The antibody can be produced by hybridoma H-215 deposited with VKPM, or can be produced by hybridoma H-216 deposited with VKPM, or can be produced by hybridoma H-217 deposited with VKPM.

In a third aspect, the present application provides an antibody or an antigen-binding fragment thereof. The antibody:
is produced based on a plasmid deposited with VKPM under a deposit number of B-14772 and a plasmid deposited with VKPM under a deposit number of B-14773;
is produced based on a plasmid deposited with VKPM under a deposit number of B-14774 and a plasmid deposited with VKPM under a deposit number of B-14775;
is produced based on a plasmid deposited with VKPM under a deposit number of B-14776 and a plasmid deposited with VKPM under a deposit number of B-14777;
is produced based on a plasmid deposited with VKPM under a deposit number of B-14778 and a plasmid deposited with VKPM under a deposit number of B-14779;
is produced based on a plasmid deposited with VKPM under a deposit number of B-14780 and a plasmid deposited with VKPM under a deposit number of B-14781;
is produced based on a plasmid deposited with VKPM under a deposit number of B-14782 and a plasmid deposited with VKPM under a deposit number of B-14783; or
is produced based on a plasmid deposited with VKPM under a deposit number of B-14784 and a plasmid deposited with VKPM under a deposit number of B-14785.

In a fourth aspect, the present application provides an isolated nucleic acid molecule encoding the antibody or the antigen-binding fragment thereof of the present application.

In a fifth aspect, the present application provides an expression vector comprising the nucleic acid molecule of the present application.

In a sixth aspect, the present application provides a host cell comprising the nucleic acid molecule of the present application or the expression vector of the present application.

In a seventh aspect, the present application provides a kit for detecting β-CTx in a sample, the kit comprising a first antibody or an antigen-binding fragment thereof and a second antibody or an antigen-binding fragment thereof, wherein the first antibody or the second antibody comprises:
a heavy chain variable region (VH) and a light chain variable region (VL),
wherein the heavy chain variable region comprises HCDR1 including an amino acid sequence as set forth in SEQ ID NO: 1, 7, 13, 19, 25, 31, 37, 43, 49 or 55 or a variant thereof, HCDR2 including an amino acid sequence as set forth in SEQ ID NO: 2, 8, 14, 20, 26, 32, 38, 44, 50 or 56 or a variant thereof, and HCDR3 including an amino acid sequence as set forth in SEQ ID NO: 3, 9, 15, 21, 27, 33, 39, 45, 51 or 57 or a variant thereof;
wherein the light chain variable region comprises LCDR1 including an amino acid sequence as set forth in SEQ ID NO: 4, 10, 16, 22, 28, 34, 40, 46, 52 or 58 or a variant thereof, LCDR2 including an amino acid sequence as set forth in SEQ ID NO: 5, 11, 17, 23, 29, 35, 41, 47, 53 or 59 or a variant thereof, and LCDR3 including an amino acid sequence as set forth in SEQ ID NO: 6, 12, 18, 24, 30, 36, 42, 48, 54 or 60 or a variant thereof; and
wherein each variant comprises one or more amino acid mutations compared to an amino acid sequence from which it is derived, and each amino acid mutation is selected from amino acid substitution, deletion or addition; preferably, the substitution is a conservative substitution; preferably, each variant has at least 80%, at least 90%, or at least 95% identity to the amino acid sequence from which it is derived.

In a specific embodiment, the sample is serum or plasma, such as heparin plasma or EDTA plasma, preferably serum or plasma collected after 8 h, 12 h, 24 h, 2 days, 4 days, or 8 days.

In a specific embodiment, the first antibody is different from the second antibody, and each of the first antibody and the second antibody comprises:
HCDR1, HCDR2, HCDR3, LCDR1, LCDR2 and LCDR3 including sequences that have at least 80%, at least 90%, at least 95%, or 100% identity to SEQ ID NOs: 1-6, respectively;
HCDR1, HCDR2, HCDR3, LCDR1, LCDR2 and LCDR3 including sequences that have at least 80%, at least 90%, at least 95%, or 100% identity to SEQ ID NOs: 7-12, respectively;
HCDR1, HCDR2, HCDR3, LCDR1, LCDR2 and LCDR3 including sequences that have at least 80%, at least 90%, at least 95%, or 100% identity to SEQ ID NOs: 13-18, respectively;
HCDR1, HCDR2, HCDR3, LCDR1, LCDR2 and LCDR3 including sequences that have at least 80%, at least 90%, at least 95%, or 100% identity to SEQ ID NOs: 19-24, respectively;
HCDR1, HCDR2, HCDR3, LCDR1, LCDR2 and LCDR3 including sequences that have at least 80%, at least 90%, at least 95%, or 100% identity to SEQ ID NOs: 25-30, respectively;
HCDR1, HCDR2, HCDR3, LCDR1, LCDR2 and LCDR3 including sequences that have at least 80%, at least 90%, at least 95%, or 100% identity to SEQ ID NOs: 31-36, respectively;
HCDR1, HCDR2, HCDR3, LCDR1, LCDR2 and LCDR3 including sequences that have at least 80%, at least 90%, at least 95%, or 100% identity to SEQ ID NOs: 37-42, respectively;
HCDR1, HCDR2, HCDR3, LCDR1, LCDR2 and LCDR3 including sequences that have at least 80%, at least 90%, at least 95%, or 100% identity to SEQ ID NOs: 43-48, respectively;
HCDR1, HCDR2, HCDR3, LCDR1, LCDR2 and LCDR3 including sequences that have at least 80%, at least 90%, at least 95%, or 100% identity to SEQ ID NOs: 49-54, respectively; or
HCDR1, HCDR2, HCDR3, LCDR1, LCDR2 and LCDR3 including sequences that have at least 80%, at least 90%, at least 95%, or 100% identity to SEQ ID NOs: 55-60, respectively.

In some embodiments, the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 55-60, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 1-6, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 25-30, respectively;
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 37-42, respectively;
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 13-18, respectively;
the first antibody comprises HCDR1, HCDR2, HCDR3, LCDR1, LCDR2 and LCDR3 including sequences as set forth in SEQ ID NOs: 13-18, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 13-18, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 19-24, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 19-24, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 25-30, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 25-30, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 31-36, respectively; and the second antibody comprises HCDR1, HCDR2, HCDR3, LCDR1, LCDR2 and LCDR3 including sequences as set forth in SEQ ID NOs: 13-18, respectively;
the first antibody comprises HCDR1, HCDR2, HCDR3, LCDR1, LCDR2 and LCDR3 including sequences as set forth in SEQ ID NOs: 31-36, respectively; and the second antibody comprises HCDR1, HCDR2, HCDR3, LCDR1, LCDR2 and LCDR3 including sequences as set forth in SEQ ID NOs: 25-30, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 13-18, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 49-54, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 19-24, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 25-30, respectively; or
the first antibody comprises HCDR1, HCDR2, HCDR3, LCDR1, LCDR2 and LCDR3 including sequences as set forth in SEQ ID NOs: 55-60, respectively; and the second antibody comprises HCDR1, HCDR2, HCDR3, LCDR1, LCDR2 and LCDR3 including sequences as set forth in SEQ ID NOs: 25-30, respectively.

By selecting a specific combination of the first antibody and the second antibody, the present application provides a kit for detecting β-CTx. The kit has a low cross-reactivity with α-CTx and has a low limit of detection (LOD).

In some embodiments, the percent cross-reactivity of the kit with α-CTx is not higher than 0.20%, such as not higher than 0.10%, 0.05%, or 0.02%.

In a specific embodiment,
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 55-60, respectively; and the second antibody comprises HCDR1, HCDR2, HCDR3, LCDR1, LCDR2 and LCDR3 including sequences as set forth in SEQ ID NOs: 1-6, respectively; or
the first antibody comprises HCDR1, HCDR2, HCDR3, LCDR1, LCDR2 and LCDR3 including sequences as set forth in SEQ ID NOs: 1-6, respectively; and the second antibody comprises HCDR1, HCDR2, HCDR3, LCDR1, LCDR2 and LCDR3 including sequences as set forth in SEQ ID NOs: 7-12, respectively.

In some embodiments,
the first antibody comprises HCDR1, HCDR2, HCDR3, LCDR1, LCDR2 and LCDR3 including sequences as set forth in SEQ ID NOs: 13-18, respectively; and the second antibody comprises HCDR1, HCDR2, HCDR3, LCDR1, LCDR2 and LCDR3 including sequences as set forth in SEQ ID NOs: 37-42, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 31-36, respectively;
the first antibody comprises HCDR1, HCDR2, HCDR3, LCDR1, LCDR2 and LCDR3 including sequences as set forth in SEQ ID NOs: 1-6, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 55-60, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 19-24, respectively; and the second antibody comprises HCDR1, HCDR2, HCDR3, LCDR1, LCDR2 and LCDR3 including sequences as set forth in SEQ ID NOs: 1-6, respectively; or
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 25-30, respectively.

In a specific embodiment, the first antibody is a capture antibody.

In a specific embodiment, the second antibody is a detection antibody.

In an eighth aspect, the present application provides a method for diagnosing osteoporosis in a subject, including:
using a first antibody or an antigen-binding fragment thereof and a second antibody or an antigen-binding fragment thereof to detect a sample from the subject;
determining the amount of β-CTx in the sample; and
determining whether the subject has osteoporosis based on the determined amount of R-CTx,
wherein one or two of the first antibody and the second antibody comprise:
a heavy chain variable region (VH) and a light chain variable region (VL),
wherein the heavy chain variable region comprises HCDR1 including an amino acid sequence as set forth in SEQ ID NO: 1, 7, 13, 19, 25, 31, 37, 43, 49 or 55 or a variant thereof, HCDR2 including an amino acid sequence as set forth in SEQ ID NO: 2, 8, 14, 20, 26, 32, 38, 44, 50 or 56 or a variant thereof, and HCDR3 including an amino acid sequence as set forth in SEQ ID NO: 3, 9, 15, 21, 27, 33, 39, 45, 51 or 57 or a variant thereof;
wherein the light chain variable region comprises LCDR1 including an amino acid sequence as set forth in SEQ ID NO: 4, 10, 16, 22, 28, 34, 40, 46, 52 or 58 or a variant thereof, LCDR2 including an amino acid sequence as set forth in SEQ ID NO: 5, 11, 17, 23, 29, 35, 41, 47, 53 or 59 or a variant thereof, and LCDR3 including an amino acid sequence as set forth in SEQ ID NO: 6, 12, 18, 24, 30, 36, 42, 48, 54 or 60 or a variant thereof; and
wherein each variant comprises one or more amino acid mutations compared to an amino acid sequence from which it is derived, and each amino acid mutation is selected from amino acid substitution, deletion or addition; preferably, each substitution is a conservative substitution; preferably, the variant has at least 80%, at least 90%, or at least 95% identity to the amino acid sequence from which it is derived.

In a specific embodiment, the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 55-60, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 1-6, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 25-30, respectively;
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 37-42, respectively;
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 13-18, respectively;
the first antibody comprises HCDR1, HCDR2, HCDR3, LCDR1, LCDR2 and LCDR3 including sequences as set forth in SEQ ID NOs: 13-18, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 13-18, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 19-24, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 19-24, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 25-30, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 25-30, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 31-36, respectively; and the second antibody comprises HCDR1, HCDR2, HCDR3, LCDR1, LCDR2 and LCDR3 including sequences as set forth in SEQ ID NOs: 13-18, respectively;
the first antibody comprises HCDR1, HCDR2, HCDR3, LCDR1, LCDR2 and LCDR3 including sequences as set forth in SEQ ID NOs: 31-36, respectively; and the second antibody comprises HCDR1, HCDR2, HCDR3, LCDR1, LCDR2 and LCDR3 including sequences as set forth in SEQ ID NOs: 25-30, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 13-18, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 49-54, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 19-24, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 25-30, respectively; or
the first antibody comprises HCDR1, HCDR2, HCDR3, LCDR1, LCDR2 and LCDR3 including sequences as set forth in SEQ ID NOs: 55-60, respectively; and the second antibody comprises HCDR1, HCDR2, HCDR3, LCDR1, LCDR2 and LCDR3 including sequences as set forth in SEQ ID NOs: 25-30, respectively.

### BRIEF DESCRIPTION OF THE DRAWINGS

FIG. 1 shows the detection consistency of Kit A (graph at top: heparin lithium VS serum; graph at bottom: EDTA-K2 VS serum).

FIG. 2 shows the detection consistency of Kit B (graph at top: heparin lithium VS serum; graph at bottom: EDTA-K2 VS serum).

FIG. 3 shows the detection consistency of Kit C (graph at top: heparin lithium VS serum; graph at bottom: EDTA-K2 VS serum).

FIG. 4 shows the detection consistency of Kit D (graph at top: heparin lithium VS serum; graph at bottom: EDTA-K2 VS serum).

FIG. 5 shows the detection consistency of Kit E (graph at top: heparin lithium VS serum; graph at bottom: EDTA-K2 VS serum).

FIG. 6 shows the detection consistency of Kit F (graph at top: heparin lithium VS serum; graph at bottom: EDTA-K2 VS serum).

FIG. 7 shows the fitting results of the measured and theoretical values of Kit A.

FIG. 8 shows the fitting results of the measured and theoretical values of Kit B.

FIG. 9 shows the fitting results of the measured and theoretical values of Kit C.

### DETAILED DESCRIPTION

The technical solutions in the embodiments of the present application are clearly and fully described below. Obviously, the described embodiments are only merely some, but not all of the embodiments of the present application. Based on the embodiments of the present application, all other embodiments obtained by those of ordinary skill in the art without creative work should fall within the scope of the present application.

Throughout the specification, unless otherwise specified, terms used herein are to be understood as meanings as commonly used in the art. Therefore, unless otherwise defined, all technical and scientific terms used herein have the same meaning as those skilled in the art to which the present application belongs. If a conflict occurs, the specification will take precedence.

As used herein, the term “antibody” refers to an immunoglobulin molecule, including but not limited to monoclonal antibodies, polyclonal antibodies, bispecific antibodies, chimeric antibodies, humanized antibodies and single-chain antibodies. In addition, it also relates to antibodies that are recombinantly or synthetically produced/synthesized.

As used herein, the term “monoclonal antibody” refers to a highly homogeneous population of antibodies only against a specific antigen epitope produced by a single B cell clone. That is, the individual antibodies that constitute the population are the same except for the possible naturally occurring mutations that may exist in small quantities. Monoclonal antibodies are highly specific for a single antigen. Monoclonal antibodies are advantageous because they can be obtained by culture of hybridoma cell lines and are essentially free from contamination by other immunoglobulins.

Monoclonal antibodies can be prepared by any of a variety of techniques known to those of ordinary skill in the art. For example, these methods may include the following steps: An immortal cell line capable of producing antibodies with the desired specificity is prepared. Such cell lines may be produced, for example, from spleen cells derived from immune animals. Spleen cells are then, immortalized, for example, by fusing with myeloma cell fusion partners (such as partners homologous to the animal being immunized). For example, membrane fusion promoters (such as polyethylene glycol or nonionic detergents) may be used to contacting spleen cells with myeloma cells for several minutes, and then plated at low density on selective medium that supports growth of hybrid cells but does not support growth of myeloma cells. After enough time, hybrid colonies are observed. Individual colonies are selected and tested for antigen binding activity. Hybridomas with high reactivity and specificity are preferred. Monoclonal antibodies may be isolated from the supernatant of the culture where hybridoma colonies grow. Contaminants may be removed from antibodies by conventional techniques such as chromatography, gel filtration, precipitation or extraction. For example, antibodies may be purified by chromatography with immobilized Protein G or Protein A using standard techniques.

Naturally occurring antibodies are produced by heavy chain only or by assembly of heavy and light chains. Each heavy chain consists of four domains: a variable region (VH), CH1, CH2 and CH3. Each light chain consists of a variable region (VL) and a constant region (CL). In the presence of heavy and light chains, the light chain is paired with a homologous heavy chain Fab fragment containing the VH and CH1 domains. The associated light and heavy chain Fab fragments are represented together as Fab fragments. The CH2 and CH3 domains represented together as the heavy chain Fc region are dimerized with the additional CH2 and CH3 domains from the second chain to form the Fc region. The Fc region is connected to the Fab fragment(s) via a flexible hinge region. The hinge region contains several disulfide bonds covalently connecting two heavy chain Fc regions together. In the Fab fragment, the light chain and the heavy chain are also connected by a disulfide bridge. However, the connectivity between IgG subclasses is different. The overall structure of full-length IgG is similar to a Y-shape, where the Fc region forms a base, while the two Fab fragments form arms and can be used to bind to an antigen.

The amino acid positions of all CDRs of the heavy and light chains of the antibodies of the present application are defined according to Kabat provided by Kabat, et al. (Sequences of Proteins of Immunological Interest, 5th ed., Public Health Service, National Institutes of Health, Bethesda, MD (1991)).

Complementarity determining regions (CDRs) are amino acid residues located within a variable region that are identified according to the Kabat definition, Chothia definition, AbM definition, contact definition and/or conformation definition or any other CDR determination method well known in the art. The CDRs of antibodies may be recognized as hypervariable regions originally defined by Kabat et al. see, for example, Kabat, et al., Sequences of Proteins of Immunological Interest, 5th ed., Public Health Service, National Institutes of Health, Bethesda, MD (1991). The locations of the CDRs may also be identified as the structural loop structure first described by Clothia et al. see, for example, Chothia et al., Nature 342:877-883, 1989. Other methods for identifying CDRs include the “AbM definition”, a compromise between Kabat and Chothia and derived from that used by Oxford Molecular's AbM antibody modeling software. “Contact definition” is based on observed antigen contacts, such as MacCallim et al., J. Mol. Biol. 262:732-745, 1996. In the “conformation definition”, the locations of CDRs may be identified as residues that make enthalpic contributions to antigen binding, see, for example, Makabe et al., Journal of Biological Chemistry, 283:1156-1166, 2008.

As used herein, the amino acid sequences as set forth in SEQ ID NOs:1-60 are defined according to Kabat. Those skilled in the art will appreciate that CDR sequences determined by different definitions are not identical and the antibodies of the present application also encompass antibodies comprising CDRs defined by any of the Chothia definition, AbM definition, contact definition and conformation definition.

As used herein, the term “antigen-binding fragment” generally includes at least a portion (e.g., six CDRs) of the antigen-binding region, light chain and/or heavy chain variable regions of the parent antibody that retains at least some of the binding specificity of the parent antibody. Examples of antigen-binding fragments include, but are not limited to, Fab, Fab′, F(ab′)2, and Fv fragments.

Preferably, the antigen-binding fragment refers to the antigen-binding region, light and heavy chain variable regions or six CDRs of an antibody.

As used herein, the term “variant” refers to a sequence that may have a substitution, deletion, and/or addition of one or more amino acid residues compared to the sequence from which it derived. The biological activities of these variants are essentially unchanged compared to the sequences from which they are derived, such as allowing the antibodies to specifically bind to β-CTx when used in constituting the antibodies of the present application. The sequence from which the variant is derived maybe the full-length sequence, heavy chain, light chain, heavy chain variable region, light chain variable region, HCDR1, HCDR2, HCDR3, LCDR1, LCDR2 and LCDR3 of the antibody. Preferably, the amino acid substitution is a conservative substitution.

As used herein, the term “conservative substitution” refers to the substitution of residues in the current amino acid sequence with other amino acid residues with similar characteristics (such as charge, side chain size, hydrophobicity/hydrophilicity, main chain conformation and rigidity) such that changes in the type of amino acid residues occur without changing the biological activity of the protein. Exemplary conservative substitutions include mutual substitutions of amino acids in each of the following eight groups of: 1) alanine (A), glycine (G); 2) aspartic acid (D), glutamic acid (E); 3) asparagine (N), glutamine (Q); 4) arginine (R), lysine (K); 5) isoleucine (I), leucine (L), methionine (M), valine (V); 6) phenylalanine (F), tyrosine (Y), tryptophan (W); 7) serine (S), threonine (T); and 8) cysteine (C), methionine (M) (see, for example, Creighton, Proteins (1984)).

In some embodiments, the variant after substitution, deletion or addition has at least 80%, at least 85%, at least 90%, at least 91%, at least 92%, at least 93%, at least 95%, at least 95%, at least 96%, at least 97%, at least 98% or at least 99% identity to the reference sequences.

As used herein, “percent (%) identity” relative to a reference polypeptide sequence refers to the percentage of amino acid residues in a variant sequence that are identical to the amino acid residues in the reference polypeptide sequence after aligning the sequences and introducing gaps if necessary to achieve the maximum percent sequence identity, and conservative substitutions are not considered identical for alignment purposes. Alignment to determine percent amino acid sequence identity can be achieved in a variety of ways within the skill of the art, for example, using publicly available computer software such as BLAST, BLAST-2, Clustal W, Megalign (DNASTAR) software or the FASTA package. Those skilled in the art can determine appropriate parameters for aligning sequences, including any algorithm needed to achieve maximum alignment over the full length of the sequences being compared. Alternatively, the sequence alignment computer program ALIGN-2 (Genentech, Inc.) may be used to produce a percent identity. The ALIGN-2 sequence alignment computer program was created by Genentech, Inc.

Preferably, substitution, deletion and addition are performed by adopting those substitutions, deletions and additions identified through alignment between the CDRs of different antibodies of the application. As described previously, alignments may be performed by BLAST, BLAST-2, Clustal W, Megalign (DNASTAR) software or the FASTA package, and the alignments may be, for example, pairwise alignments, or alignments between more CDRs. As an illustrative example, by alignment between the HCDR2 of the antibody b-CTx_R_rec_145 and the antibody b-CTx_R_rec_138, the mutation are substitutions of S6G, G7D and T9I.

In some embodiments, the antibody or the antigen-binding fragment thereof of the present application specifically binds to AH(betaD)G.

In some embodiments, the antibody or the antigen-binding fragment thereof of the present application specifically binds to AH(betaD)GG.

In some embodiments, the antibody or the antigen-binding fragment thereof of the present application specifically binds to AH(betaD)GGR.

In some embodiments, the antibody or the antigen-binding fragment thereof of the present application specifically binds to AH(betaD)GGR-COOH.

In other embodiments, the antibody or the antigen-binding fragment thereof of the present application specifically binds to more than one of AH(betaD)G, AH(betaD)GG, AH(betaD)GGR and AH(betaD)GGR-COOH. In one exemplary embodiment, the antibody or the antigen-binding fragment thereof of the present application specifically binds to AH (betaD)GG and AH (betaD)G.

The present application relates to: the hybridoma cell deposited with the Russian National Collection of Industrial Microorganisms (VKPM) (Research Centre “Kurchatov Institute” State Research Institute “Genetika” 1-st Dorozhniy pr., 1 Moscow 117545, Russian Federation) on Mar. 28, 2024 under a deposit number of H-215, and the antibody b-CTx_M_hyb_015 produced by the hybridoma cell; antibodies comprising the heavy chain and light chain of the antibody produced by the hybridoma cell; antibodies comprising the heavy chain variable region and light chain variable region of the antibody produced by the hybridoma cell; and antibodies having 80%, at least 85%, at least 90%, at least 91%, at least 92%, at least 93%, at least 94%, at least 95%, at least 96%, at least 97%, at least 98% or at least 99% identity to the amino acid sequence of b-CTx_M_hyb_015. Preferably, the change caused by % identity does not occur at the CDRs or does not occur at the heavy chain variable region and the light chain variable region.

The present application relates to: the hybridoma cell deposited with the Russian National Collection of Industrial Microorganisms (VKPM) on Mar. 28, 2024 under a deposit number of H-216; the antibody b-CTx_M_hyb_061 produced by the hybridoma cell; antibodies comprising the heavy chain and light chain of the antibody produced by the hybridoma cell; antibodies comprising the heavy chain variable region and light chain variable region of the antibody produced by the hybridoma cell; to antibodies having 80%, at least 85%, at least 90%, at least 91%, at least 92%, at least 93%, at least 94%, at least 95%, at least 96%, at least 97%, at least 98% or at least 99% identity to the amino acid sequence of b-CTx_M_hyb_061. Preferably, the change caused by % identity does not occur at the CDRs or does not occur at the heavy chain variable region and the light chain variable region.

The present application relates to: the hybridoma cell deposited with the Russian National Collection of Industrial Microorganisms (VKPM) on Mar. 28, 2024 under a deposit number of H-217; the antibody b-CTx_M_hyb_063 produced by the hybridoma cell; antibodies comprising the heavy chain and light chain of the antibody produced by the hybridoma cell; antibodies comprising the heavy chain variable region and light chain variable region of the antibody produced by the hybridoma cell; and antibodies having 80%, at least 85%, at least 90%, at least 91%, at least 92%, at least 93%, at least 94%, at least 95%, at least 96%, at least 97%, at least 98% or at least 99% identity to the amino acid sequence of b-CTx_M_hyb_063. Preferably, the change caused by % identity does not occur at the CDRs or does not occur at the heavy chain variable region and the light chain variable region.

The present application relates to the cell deposited with VKPM on Apr. 2, 2024, under a deposit number of B-14772, a plasmid carried by the cell, and a light chain produced by the plasmid; and relates to the cell deposited with VKPM on Apr. 2, 2024 under a deposit number of B-14773, a plasmid carried by the cell, and a heavy chain produced by the plasmid. Further, the antibody b-CTx_R_rec_132 is provided, which comprises the light chain produced by the plasmid in B-14772 and the heavy chain produced by the plasmid in B-14773. Further provided is an antibody, which has 80%, at least 85%, at least 90%, at least 91%, at least 92%, at least 93%, at least 94%, at least 95%, at least 96%, at least 97%, at least 98% or at least 99% identity to the amino acid sequences of the heavy chain and light chain of b-CTx_R_rec_132. Preferably, the change caused by % identity does not occur at the CDRs or does not occur at the heavy chain variable region and the light chain variable region. Further provided is an antibody, which has the same heavy chain variable region and light chain variable region as b-CTx_R_rec_132.

The present application relates to the cell deposited with VKPM on Apr. 2, 2024, under a deposit number of B-14774, a plasmid carried by the cell, and a light chain produced by the plasmid; and relates to the cell deposited with VKPM on Apr. 2, 2024 under a deposit number of B-14775, a plasmid carried by the cell, and a heavy chain produced by the plasmid. Further, the antibody b-CTx_R_rec_134 is provided, which comprises the light chain produced by the plasmid in B-14774 and the heavy chain produced by the plasmid in B-14775. Further provided is an antibody, which has 80%, at least 85%, at least 90%, at least 91%, at least 92%, at least 93%, at least 94%, at least 95%, at least 96%, at least 97%, at least 98% or at least 99% identity to the amino acid sequences of the heavy chain and light chain of b-CTx_R_rec_134. Preferably, the change caused by % identity does not occur at the CDRs or does not occur at the heavy chain variable region and the light chain variable region. Further provided is an antibody, which has the same heavy chain variable region and light chain variable region as b-CTx_R_rec_134.

The present application relates to the cell deposited with VKPM on Apr. 2, 2024, under a deposit number of B-14776, a plasmid carried by the cell, and a light chain produced by the plasmid; and relates to the cell deposited with VKPM on Apr. 2, 2024 under a deposit number of B-14777, a plasmid carried by the cell, and a heavy chain produced by the plasmid. Further, the antibody b-CTx_R_rec_137 is provided, which comprises the light chain produced by the plasmid in B-14776 and the heavy chain produced by the plasmid in B-14777. Further provided is an antibody, which has 80%, at least 85%, at least 90%, at least 91%, at least 92%, at least 93%, at least 94%, at least 95%, at least 96%, at least 97%, at least 98% or at least 99% identity to the amino acid sequences of the heavy chain and light chain of b-CTx_R_rec_137. Preferably, the change caused by % identity does not occur at the CDRs or does not occur at the heavy chain variable region and the light chain variable region. Further provided is an antibody, which has the same heavy chain variable region and light chain variable region as b-CTx_R_rec_137.

The present application relates to the cell deposited with VKPM on Apr. 2, 2024, under a deposit number of B-14778, a plasmid carried by the cell, and a light chain produced by the plasmid; and to the cell deposited with VKPM on Apr. 2, 2024 under a deposit number of B-14779, a plasmid carried by the cell, and a heavy chain produced by the plasmid. Further, the antibody b-CTx_R_rec_138 is provided, which comprises the light chain produced by the plasmid in B-14778 and the heavy chain produced by the plasmid in B-14779. Further provided is an antibody, which has 80%, at least 85%, at least 90%, at least 91%, at least 92%, at least 93%, at least 94%, at least 95%, at least 96%, at least 97%, at least 98% or at least 99% identity to the amino acid sequences of the heavy chain and light chain of b-CTx_R_rec_138. Preferably, the change caused by % identity does not occur at the CDRs or does not occur at the heavy chain variable region and the light chain variable region. Further provided is an antibody, which has the same heavy chain variable region and light chain variable region as b-CTx_R_rec_138.

The present application relates to the cell deposited with VKPM on Apr. 2, 2024, under a deposit number of B-14780, a plasmid carried by the cell, and a light chain produced by the plasmid; and relates to the cell deposited with VKPM on Apr. 2, 2024 under a deposit number of B-14781, a plasmid carried by the cell, and a heavy chain produced by the plasmid. Further, the antibody b-CTx_R_rec_145 is provided, which comprises the light chain produced by the plasmid in B-14780 and the heavy chain produced by the plasmid in B-14781. Further provided is an antibody, which has 80%, at least 85%, at least 90%, at least 91%, at least 92%, at least 93%, at least 94%, at least 95%, at least 96%, at least 97%, at least 98% or at least 99% identity to the amino acid sequences of the heavy chain and light chain of b-CTx_R_rec_145. Preferably, the change caused by % identity does not occur at the CDRs or does not occur at the heavy chain variable region and the light chain variable region. Further provided is an antibody, which has the same heavy chain variable region and light chain variable region as b-CTx_R_rec_145.

The present application relates to the cell deposited with VKPM on Apr. 2, 2024, under a deposit number of B-14782, a plasmid carried by the cell, and a light chain produced by the plasmid; and relates to the cell deposited with VKPM on Apr. 2, 2024 under a deposit number of B-14783, a plasmid carried by the cell, and a heavy chain produced by the plasmid. Further, the antibody b-CTx_R_rec_150 is provided, which comprises the light chain produced by the plasmid in B-14782 and the heavy chain produced by the plasmid in B-14783. Further provided is an antibody, which has 80%, at least 85%, at least 90%, at least 91%, at least 92%, at least 93%, at least 94%, at least 95%, at least 96%, at least 97%, at least 98% or at least 99% identity to the amino acid sequences of the heavy chain and light chain of b-CTx_R_rec_150. Preferably, the change caused by % identity does not occur at the CDRs or does not occur at the heavy chain variable region and the light chain variable region. Further provided is an antibody, which has the same heavy chain variable region and light chain variable region as b-CTx_R_rec_150.

The present application relates to the cell deposited with VKPM on Apr. 2, 2024, under a deposit number of B-14784, a plasmid carried by the cell, and a light chain produced by the plasmid; and relates to the cell deposited with VKPM on Apr. 2, 2024 under a deposit number of B-14785, and a heavy chain produced by the cell. Further, the antibody b-CTx_R_rec_191 is provided, which comprises the light chain produced by the plasmid in B-14784 and the heavy chain produced by the plasmid in B-14785. Further provided is an antibody, which has 80%, at least 85%, at least 90%, at least 91%, at least 92%, at least 93%, at least 94%, at least 95%, at least 96%, at least 97%, at least 98% or at least 99% identity to the amino acid sequences of the heavy chain and light chain of b-CTx_R_rec_191. Preferably, the change caused by % identity does not occur at the CDRs or does not occur at the heavy chain variable region and the light chain variable region. Further provided is an antibody, which has the same heavy chain variable region and light chain variable region as b-CTx_R_rec_191.

In some embodiments, the KD of the antibodies of the present application is at least less than 10⁻⁹ M, especially 10⁻¹⁰ M.

As used herein, the term “nucleic acid molecule” or “polynucleotide” includes any compound and/or substance that comprises a polymer of nucleotides. Each nucleotide consists of a base, especially a purine or pyrimidine base (i.e., cytosine (C), guanine (G), adenine (A), thymine (T) or uracil (U)), a sugar (i.e., deoxyribose or ribose), and a phosphate group. Generally, a nucleic acid molecule is described by a sequence of bases, where the bases represent the primary structure (linear structure) of the nucleic acid molecule. The sequence of bases is usually expressed as being from 5′ to 3′. As used herein, the term nucleic acid molecule includes deoxyribonucleic acid (DNA), including, for example, complementary DNA(cDNA) and genomic DNA; ribonucleic acid (RNA), especially messenger RNA(mRNA), synthetic forms of DNA or RNA, and mixed polymers containing two or more of these molecules. Nucleic acid molecules may be linear or circular. In addition, the term “nucleic acid molecule” includes sense and/or antisense strands, as well as single and double stranded forms. In addition, the nucleic acid molecules described herein may comprise naturally occurring or non-naturally occurring nucleotides. Examples of non-naturally occurring nucleotides include modified nucleotide bases with derivatized sugar or phosphate backbone bonds or chemically modified residues. Nucleic acid molecules also include DNA and RNA molecules suitable as vectors for direct expression of the antibodies of the present application in vitro and/or in vivo, for example, in a host or patient. Such DNA (e.g., cDNA) or RNA (e.g., mRNA) carriers may be unmodified or modified. For example, mRNA can be chemically modified to enhance the stability of the RNA vector and/or expression of the encoding molecule so that the mRNA can be injected into a subject to produce antibodies in the subject.

In a specific embodiment, the nucleic acid molecule of the present application encodes the antibody or the antigen-binding fragment thereof of the present application.

In a specific embodiment, the nucleic acid molecule of the present application encodes HCDR and LCDR. For example, the sequences of the nucleic acid molecules are as set forth in SEQ ID NOs: 61-66, SEQ ID NOs: 67-72, SEQ ID NOs: 73-78, SEQ ID NOs: 79-84, SEQ ID NOs: 85-90, SEQ ID NOs: 91-96, SEQ ID NOs: 97-102, SEQ ID NOs: 103-108, SEQ ID NOs: 109-114 or SEQ ID NOs: 115-120.

An “isolated” nucleic acid molecule refers to a nucleic acid molecule that has been separated from components of its natural environment. The isolated nucleic acid molecules include nucleic acid molecules contained in cells. The cells typically contain nucleic acid molecules but the nucleic acid molecules are present extra chromosomally or at chromosomal locations that are different from their natural chromosomal locations.

The “isolated nucleic acid molecule” encoding an antibody refers to one or more nucleic acid molecules encoding heavy and light chains (or antigen-binding fragments) of an antibody, including such nucleic acid molecule(s) in a single vector or separate vectors, and such nucleic acid molecule(s) present at one or more locations in a host cell.

As used herein, the term “vector” refers to a nucleic acid molecule capable of proliferating another nucleic acid to which it is attached. Vectors include vectors that are self-replicating nucleic acid structures and vectors that are incorporated into the genome of the host cell into which they have been introduced. Certain vectors are capable of directing the expression of the nucleic acid molecules to which they are operably linked. Such vectors are referred to herein as “expression vectors”.

The terms “host cell”, “host cell line” and “host cell culture” are used interchangeably and refer to cells into which foreign nucleic acid has been introduced, including the descendants of such cells. Host cells include “transformants” and “transformed cells”, which include the primary transformed cells and the progeny derived therefrom, regardless of the number of passages. The progeny may not be identical in nucleic acid content to the parent cell, but may contain mutations. Included herein are mutant progeny with the same function or biological activity as screened or selected in the originally transformed cells.

In the present application, the expressions “first”, “second”, “third”, etc. are used for descriptive purposes only to distinguish defined substances, and do not define order or priority in any way.

The kits of the present application may take a variety of forms, such as test kits for various reagents required for immunoassay kits, microfluidic chips, etc., and the kits may be manufactured according to standard procedures known to those skilled in the art.

The kit of the present application may comprise containers, solid phase carriers for immunoassay, a first antibody, a second antibody, instructions for use, buffers and/or other materials, structures and/or reagents needed for diagnosis/detection as needed.

The kit of the present application includes the antibody of the present application and may be present in a manner conventional in the art. In some embodiments, the antibody of the present application is present in a container in a dissolved or dried form, or coated on a solid carrier (such as membrane, plate, beads (such as magnetic beads and SA magnetic beads), and microspheres (such as carboxyl microspheres, amino microspheres, chloromethyl microspheres, and physically adsorbed microspheres)). In this case, the antibody of the present application may be referred to as a capture antibody.

Other reagents required for diagnosis/detection in the kits of the present application include, but are not limited to, other anti-β-CTx antibodies in addition to the antibodies of the present application, anti-rabbit antibodies, anti-mouse antibodies, etc. The above-mentioned other reactants may exist in a manner conventional in the art, for example, in a dissolved or dried form in a container, coated on a solid support, and in a dissolved or dried form in a chamber of the chip, but the present application is not limited thereto.

In specific embodiments, the components in the kit of the present application may exist independently. Depending on the different assay methods, the kit, for example, exists in the form of a first reagent and a second reagent; or exists in the form of a first reagent, a second reagent and a third reagent.

In specific embodiments, the measurement procedures of the kit are described in the instructions for use.

In some embodiments, the kit of the present application further includes a β-CTx standard. In one exemplary embodiment, the standard may be serially diluted. In specific embodiments, β-CTx antigen may be diluted to concentration points of 0 ng/mL, 0.07 ng/mL, 0.18 ng/mL, 0.32 ng/mL, 0.48 ng/mL, 0.82 ng/mL, 1.34 ng/mL, 2.81 ng/mL, 3.86 ng/mL, 5.62 ng/mL, and 6.57 ng/mL.

Other materials required for testing in the kit of the present application include, but are not limited to, materials for sampling, materials for conducting controls, and/or materials for observing the testing process or results.

Other reagents required for testing in the kit of the present application include, but are not limited to, detergents, color developers and/or terminating agents.

In some embodiments, the antibodies in the kit of the present application are labeled with a detectable label. Any label and labeling method known to those skilled in the art may be used. Commonly used labels may include enzymes (such as horseradish peroxidase, β-galactosidase, alkaline phosphatase), radioactive isotopes (such as 32P or ¹²⁵I), biotin, digoxin, colloidal metals (such as colloidal gold), fluorescent dyes (such as fluorescein, rhodamine, Texas Red), chemiluminescent compounds or bioluminescent compounds (such as dioxetane, acridinium ester, luminol, isoluminol, acridinium, tripyridinium ruthenium). Any labeling step well known in the art may be used, such as covalent coupling of an enzyme or biotin group, iodination, phosphorylation, biotinylation. In this case, the antibody of the present application may be referred to as a detection antibody.

In some embodiments, one or more of the other reactants required for testing may also be labeled with a detectable label.

In some embodiments, the kit of the present application is an immunoassay kit based on a sandwich method, an indirect method, or a competitive method.

In addition, among the current mainstream β-CTx testing methods, high freshness of test samples is required for β-CTx, and the test needs to be completed within 4-8 h after sample collection. This requirement is not conducive to the operating habits of laboratory doctors and the requirements for retest. In order to meet the operating habits of laboratory doctors and the requirements for retest, the following improvements are necessary:
1. For serum samples, the storage time of serum at room temperature or 2-8° C. needs to be extended to at least 24 h to better serve testing and clinical applications. Therefore, the prior art methods may not meet this specific testing needs.
2. For heparin plasma or EDTA plasma samples, the storage time of serum at room temperature or 2-8° C. also needs to be extended to at least 24 h to better serve testing and clinical applications. Therefore, the prior art methods may not meet this specific testing needs.
3. Consistency between test results needs to be maintained regardless of serum or plasma samples.

To this end, the present application provides a kit comprising a first antibody and a second antibody, and
the first antibody comprises HCDR1, HCDR2, HCDR3, LCDR1, LCDR2 and LCDR3 having sequences as set forth in SEQ ID NOs: 13-18, respectively; and the second antibody comprises HCDR1, HCDR2, HCDR3, LCDR1, LCDR2 and LCDR3 having sequences as set forth in SEQ ID NOs: 37-42, respectively;
the first antibody comprises HCDR1, HCDR2, HCDR3, LCDR1, LCDR2 and LCDR3 having sequences as set forth in SEQ ID NOs: 49-54, respectively; and the second antibody comprises HCDR1, HCDR2, HCDR3, LCDR1, LCDR2 and LCDR3 having sequences as set forth in SEQ ID NOs: 31-36, respectively;
the first antibody comprises HCDR1, HCDR2, HCDR3, LCDR1, LCDR2 and LCDR3 having sequences as set forth in SEQ ID NOs: 1-6, respectively; and the second antibody comprises HCDR1, HCDR2, HCDR3, LCDR1, LCDR2 and LCDR3 having sequences as set forth in SEQ ID NOs: 7-12, respectively;
the first antibody comprises HCDR1, HCDR2, HCDR3, LCDR1, LCDR2 and LCDR3 having sequences as set forth in SEQ ID NOs: 55-60, respectively; and the second antibody comprises HCDR1, HCDR2, HCDR3, LCDR1, LCDR2 and LCDR3 having sequences as set forth in SEQ ID NOs: 7-12, respectively;
the first antibody comprises HCDR1, HCDR2, HCDR3, LCDR1, LCDR2 and LCDR3 having sequences as set forth in SEQ ID NOs: 19-24, respectively; and the second antibody comprises HCDR1, HCDR2, HCDR3, LCDR1, LCDR2 and LCDR3 having sequences as set forth in SEQ ID NOs: 1-6, respectively; or
the first antibody comprises HCDR1, HCDR2, HCDR3, LCDR1, LCDR2 and LCDR3 having sequences as set forth in SEQ ID NOs: 43-48, respectively; and the second antibody comprises HCDR1, HCDR2, HCDR3, LCDR1, LCDR2 and LCDR3 having sequences as set forth in SEQ ID NOs: 25-30, respectively.

The present application also includes a method for detecting the presence of lipoprotein (a) in a subject, the method comprising: contacting a biological sample from the subject with at least one antibody or antigen-binding fragment thereof of the present application to determine the presence, in the biological sample, of a molecule that is naturally present in the sample in a soluble form and has an antigenic determinant that is reactive with the at least one antibody or antigen-binding fragment thereof, the contacting being carried out under conditions and for a time sufficient to detect binding of the antibody or antigen-binding fragment thereof to the antigenic determinant.

In one embodiment, the antibody or the antigen binding fragment thereof is detectably labeled. In another embodiment, the antibody or the antigen-binding fragment thereof is not detectably labeled, and wherein the detection of binding of the antibody or the antigen-binding fragment thereof to an antigenic determinant is indirect.

Immunoassay testing methods include but are not limited to autoradiography, turbidimetry, fluorescence microscopy, direct and indirect enzymatic reactions, radioisotope methods or non-radioisotope methods, etc. These methods specifically include, immunoturbidimetry, latex-enhanced immunoturbidimetry, Western blotting, overlay assays, RIA (radioimmunoassay) and IRMA (immunoradioimmunoassay), GIA (colloidal gold immunoassay), EIA (enzyme immunoassay), ELISA (enzyme-linked immunosorbent assay), FIA (fluorescent immunoassay), and CLIA (chemiluminescent immunoassay).

Osteoporosis is a systemic bone disease characterized by low bone mass, destruction of bone microstructure, increased bone fragility, and susceptibility to fractures. Its clinical manifestations include bone pain, spinal deformation, fractures, back curvature, etc. The antibody or the antigen-binding fragment thereof or the kit described herein are used for diagnosing osteoporosis.

In one aspect, provided is use of the antibody or the antigen-binding fragment thereof of the present application, the kit of the present application, the antibody combination of the present application in the preparation of a kit for diagnosing osteoporosis.

In one aspect, provided is use of the antibody or the antigen-binding fragment thereof of the application, the kit of the present application, the antibody combination of the application in the preparation of a kit for detecting β-CTx.

In one aspect, the application provides a composition comprising one or more of the antibodies of the present application.

In some embodiments, the composition of the present application further comprises reagents that detect one or more of the group consisting of: osteocalcin, vitamin D, P1NP and PTH.

In some embodiments, the LOD of the kit, the antibody or the binding fragment of the antibody, or the composition, described herein, for detecting β-CTx is less than 11 μg/ml, less than 10 μg/ml, less than 9 μg/ml, less than 8 μg/ml, less than 7 μg/ml, less than 6 μg/ml, less than 5 μg/ml, less than 4 μg/ml, or less than 3.5 μg/ml.

In some embodiments, the cross-reactivity of the kit or the antibody or the antibody-binding fragment thereof, or the composition, described herein, for detecting β-CTx with α-CTx is less than or equal to 0.20%, less than or equal to 0.15%, less than or equal to 0.10%, less than or equal to 0.09%, less than or equal to 0.08%, less than or equal to 0.07%, less than or equal to 0.06%, less than or equal to 0.05%, less than or equal to 0.04%, less than or equal to 0.03%, less than or equal to 0.02%, or less than or equal to 0.01%.

The kit, or the antibody or the antibody-binding fragment thereof, or the composition, described herein, is used for testing a serum sample or a plasma sample.

As used herein, the serum sample may be, for example, serum stored at 20˜25° C. for up to 4 h, 6 h, 8 h, 12 h or 16 h; or, for example, serum stored at 20˜25° C. for more than 4 h, more than 6 h, more than 8 h, or more than 12 h. The serum sample may be, for example, serum stored at 2˜8° C. for up to 4 h, 6 h, 8 h, 12 h, 24 h, or 2 days; or, for example, serum stored at 2˜8° C. for more than 4 h, more than 6 h, more than 8 h, more than 10 h, more than 12 h, more than 16 h, or more than 20 h.

As used herein, the plasma sample may be heparin plasma or EDTA plasma.

The heparin plasma may be, for example, plasma stored at 20˜25° C. for up to 4 h, 6 h, 8 h, 12 h or 16 h; or, for example, plasma stored at 20˜25° C. for more than 4 h, more than 6 h, more than 8 h, or more than 12 h. The heparin plasma may be, for example, plasma stored at 2˜8° C. for up to 4 h, 6 h, 8 h, 12 h, 24 h, or 2 days; or, for example, plasma stored at 2˜8° C. for more than 4 h, more than 6 h, more than 8 h, more than 10 h, more than 12 h, more than 16 h, or more than 20 h.

EDTA plasma may be, for example, plasma stored at 20˜25° C. for up to 4 h, 6 h, 8 h, 12 h, 16 h, 24 h or 2 days; or, for example, plasma stored at 20˜25° C. for more than 4 h, more than 6 h, more than 8 h, more than 12 h, more than 16 h, or more than 24 h. The EDTA plasma may be, for example, plasma stored at 2˜8° C. for 1 day, 2 days, 4 days, 8 days or 10 days; or, for example, plasma stored at 2˜8° C. for more than 2 days, more than 4 days or more than 8 days.

The implementation of the present application will be described in detail below in conjunction with examples. If specific conditions are not specified in the examples, routine conditions or conditions suggested by the manufacturer will be followed. The reagents or instruments used without indicating manufacturers are all conventional products that can be purchased commercially.

### Example 1 Generation of Monoclonal Antibodies Specific for Human Type I Collagen β-C-Terminal Telopeptide (β-CTx)

Recombinant human Type I collagen β-C-terminal telopeptide (β-CTx) was expressed in E. Coli and further purified. The resulting purified protein solution was concentrated, stored at −80° C. or below, and its purity was confirmed by SDS-PAGE and molecular sieve chromatography (SEC).

Immunization of mice was performed using β-CTx. The hybridoma cells obtained post-immunization were subsequently screened, to obtain hybridoma cells producing human Type I collagen β-C-terminal telopeptide (β-CTx).

Three mouse hybridomas were selected and deposited with VKPM.

The hybridoma cell line that produces b-CTx_M_hyb_015 was deposited with VKPM on Mar. 28, 2024 under a deposit number of H-215;
the hybridoma cell line that produces b-CTx_M_hyb_061 was deposited with VKPM on Mar. 28, 2024 under a deposit number of H-216;
and the hybridoma cell line that produces b-CTx_M_hyb_063 was deposited with VKPM on Mar. 28, 2024 under a deposit number of H-217.

In addition, 7 rabbit hybridomas were obtained by a similar method.

### Example 2 Sequencing of Mouse-Derived Antibodies

Total RNA was extracted from selected hybridoma cells using the Quick-RNA Microprep Kit (#1050-51, Zymo Research, USA), and the antibodies produced were sequenced according to the instructions for the kit. The sequencing results of HCDRs and LCDRs are listed in Tables 1 and 2, respectively.

TABLE 1

| Name of | | Heavy chain | | |
|---|---|---|---|---|
| Antibody | | CDR1 | CDR2 | CDR3 |
| b- | nt | AGCACTTTTGGTA | CACATTTACTGGGATGATGTCAAG | GCTCGAACCGCTGGGGCCT |
| CTx_M_ | | TGGGTGTGAGC | CGCTATAATCCATCCCTGAAGAGC | GGTTTGCTTTC |
| hyb_015 | aa | STFGMGVS | HIYWDDVKRYNPSLKS | ARTAGAWFAF |
| b- | nt | AGCACTTATGGTA | CACATTTGGTGGGATGATGTCAAG | GCTCGCATACCTGGGGATG |
| CTx_M_ | | TGGGTATAGGC | CGCTATGATCCAGTCCTGAAGAGC | GTTCCCACAGTGACTAC |
| hyb_061 | aa | STYGMGIG | HIWWDDVKRYDPVLKS | ARIPGDGSHSDY |
| b- | nt | AGCACTTCTGGTA | CACATTTGGTGGGATGATGTCAAG | GCTCGAATACCTGGGGATG |
| CTx_M | | TGGGTATAGGC | CGCTATGATCCAGCCCTGAAGAGC | GTTCCCACTTTGACTAC |
| hyb_063 | aa | STSGMGIG | HIWWDDVKRYDPALKS | ARIPGDGSHFDY |

TABLE 2

| Name of | | Light chain | | |
|---|---|---|---|---|
| Antibody | | CDR1 | CDR2 | CDR3 |
| b- | nt | AGTGCCAGGTCAACTGTAAATTAC | GACACATCCAAAC | CAGCAGTGGACTAGTAATT |
| CTx_M_ | | ATGCAC | TGGCTTCT | TGCTCACG |
| hyb_015 | aa | SARSTVNYMH | DTSKLAS | QQWTSNLLT |
| (kappa) | | | | |
| b- | nt | AGATCTACTCAGAGCCTTGTACAT | AGAATTTCCAACC | TTTCAAGGTTCACATGGTC |
| CTx_M_ | | AGTAATGGAATCACCTATTTAGAG | GATTTTCT | CGTACACG |
| hyb_061 | aa | RSTQSLVHSNGITYLE | RISNRFS | FQGSHGPYT |
| (kappa) | | | | |
| b- | nt | AGATCTAGTCAGAGCCTTGTACAT | AGAGTTTCCAACC | TTTCAAGGTTCACATGGTC |
| CTx_M_ | | AGTAATGGAATCACCTATTTAGAG | GATTTTCT | CGTACACG |
| hyb_063 | aa | RSSQSLVHSNGITYLE | RVSNRFS | FQGSHGPYT |
| (kappa) | | | | |

### Example 3 Sequencing of Rabbit-Derived Antibodies

Total RNA was extracted from approximately 500 hybridoma cells using Norgen Single Cell Purification Kit (#51800, Norgen Biotek, Canada), and the antibodies produced were sequenced according to the instructions for the kit. The sequencing results of HCDRs and LCDRs are listed in Tables 3 and 4, respectively.

TABLE 3

| Name of | | Heavy chain | | |
|---|---|---|---|---|
| Antibody | | CDR1 | CDR2 | CDR3 |
| b- | nt | AGTAGCGC | TACATTTATGCTGGTAGTGCTA | GCCAGAAGTTTGAGTG |
| CTx_R_ | | TGCAATAA | GCACATACTACGCGACCTGGG | GTGGTGATCGGAACTT |
| rec 132 | | AC | CGAAAGGC | G |
| | aa | SSAAIN | YIYAGSASTYYATWAKG | ARSLSGGDRNL |
| b- | nt | AGTAGCGC | TACATTTATGCTGGTAGTGGTA | GCCAGAAGTTTGAGTG |
| CTx_R_ | | TGCAATAA | GCACATACTACGCGAGCTGGG | GCGGTGATCGGAACTT |
| rec_134 | | AT | CGAAAGGC | G |
| | aa | SSAAIN | YIYAGSGSTYYASWAKG | ARSLSGGDRNL |
| b- | nt | AGTAGCGC | TACATTTATGCTGGTAGTGGTA | GCCAGAAGTTTGAGTG |
| CTx_R_ | | TGCAATAA | GCACATACTACGCGAGCTGGG | GCGGTGATCGGAACTT |
| rec_137 | | AT | CGAAAGGC | G |
| | aa | SSAAIN | YIYAGSGSTYYASWAKG | ARSLSGGDRNL |
| b- | nt | AGTAGCGC | TACATTTATGCTGGTGATGATA | GCCAGAAGTTTGAGTG |
| CTx_R_ | | TGCAATAA | GTATATACTACGCGAGCTGGG | GTGGTGATCGGAACTT |
| rec_138 | | AC | CGAAAGGC | G |
| | aa | SSAAIN | YIYAGDDSIYYASWAKG | ARSLSGGDRNL |
| b- | nt | AGTAGCGC | TACATTTATGCTGGTAGTGGTA | GTCAGAAGTTTAAGTG |
| CTx_R_ | | TGCAATAA | GCACATACTACGCGAGCTGGG | GTGGTGATCGGAACTT |
| rec_145 | | AC | CGAAAGGC | G |
| | aa | SSAAIN | YIYAGSGSTYYASWAKG | VRSLSGGDRNL |
| b- | nt | AGTAAATA | TGGATTACTAGTGATGGTATCA | ACCAGAAGTATACTTA |
| CTx_R_ | | TGGAGTGA | CATTCTACGCGAGCTGGGCGA | GTGCTTGGGATAACAT |
| rec_150 | | AC | AAGGC | C |
| | aa | SKYGVN | WITSDGITFYASWAKG | TRSILSAWDNI |
| b- | nt | AGTAGCTA | GGCATTAATACTGGTGGTGTC | GCCAGACATCCTGGTG |
| CTx_R_ | | CTACGTGA | GCATACTACGCGACCTGGGCA | GTGATGCTGGTACCGA |
| rec_191 | | GC | AAAGGC | CTTG |
| | aa | SSYYVS | GINTGGVAYYATWAKG | ARHPGGDAGTDL |

TABLE 4

| Name of | | Light chain (with all of Kappa) | | |
|---|---|---|---|---|
| Antibody | | CDR1 | CDR2 | CDR3 |
| b- | nt | CAGTCCAGTGAGAGTG | CTCCTGATCTACG | CAAGGCGGTTATAGTAG |
| CTx_R_ | | TTACTAAGAACAACTA | AAGCATCCAAAC | GAATAATGATATGACT |
| rec_132 | | CTTAGCC | TGGCA | |
| kappa | aa | QSSESVTKNNYLA | LLIYEASKLA | QGGYSRNNDMT |
| b- | nt | CAGTCCAGTGAGAATG | CTCCTGATCTACG | CAAGGCGGTTATAATAG |
| CTx_R_ | | TTGCTAAGAACAACTA | AAGCATCCAAAC | GAATAATGATATGACT |
| rec_134 | | CTTAGCC | TGGCA | |
| kappa | aa | QSSENVAKNNYLA | LLIYEASKLA | QGGYNRNNDMT |
| b- | nt | CAGTCCAGTGAGAGTG | CTCCTGATCTACG | CAAGGCGGTTATAGTAG |
| CTx_R_ | | TTTCTAAGAACAACTA | AAGCATCCAAAC | GAATAATGATATGACT |
| rec_137 | | CTTAGCC | TGGCA | |
| kappa | aa | QSSESVSKNNYLA | LLIYEASKLA | QGGYSRNNDMT |
| b- | nt | CAGTCCAGTGAGAGTG | CTCCTGATCTACG | CAAGGCGGTTATAGTAG |
| CTx_R_ | | TTTCTAAGAACAACTA | AAGCATCCAAAC | GAATAATGATATGACT |
| rec_138 | | CTTAGCC | TGCCA | |
| kappa | aa | QSSESVSKNNYLA | LLIYEASKLP | QGGYSRNNDMT |
| b- | nt | CAGTCCAGTGAGAGTG | CTCCTGATCTACA | CAAGGCGGTTATAGTAG |
| CTx_R_ | | TTTCTAAGAATGATTAC | GGGCTTCCACTC | GAATAATGATATGACT |
| rec_145 | | TTAGCC | TGGCA | |
| kappa | aa | QSSESVSKNDYLA | LLIYRASTLA | QGGYSRNNDMT |
| b- | nt | CAGTCCAGTAAGAGTG | CGCCTGATCTATT | GCAGGCGGTTATAGTGG |
| CTx_R_ | | TTTATAATGGGAACTG | CTGCATCCACTCT | TGAGATTGATGAT |
| rec_150 | | GTTATCC | GGCA | |
| kappa | aa | QSSKSVYNGNWLS | RLIYSASTLA | AGGYSGEIDD |
| b- | nt | CAGGCCAGTCAGAGC | CGCCTGATCTACA | CAAAACAATGTTCCTGG |
| CTx_R_ | | ATTACTAAATACTTAGC | AGACATCCACTC | TAAAATTACTGACAGTTA |
| rec_191 | | C | TGGCA | TGGGGCT |
| kappa | aa | QASQSITKYLA | RLIYKTSTLA | QNNVPGKITDSYGA |

### Example 4 Production and Purification of Antibodies

Hybridoma cells H-125, H-216 and H-217 were cultured in vitro, and antibodies b-CTx_M_hyb_015, b-CTxM_hyb_061 and b-CTx_M_hyb_063 were isolated using conditioned medium for cell culture.

Rabbit antibodies b-CTx_R_rec_132, b-CTx_R_rec_134, b-CTx_R_rec_137, b-CTx_R_rec_138, b-CTx_R_rec_145, b-CTx_R_rec_150 and b-CTx_R_rec_191 were produced in recombinant forms. Plasmids encoding the amino acid sequences of the light chain and heavy chain of the recombinant antibody were obtained from the 7 rabbit hybridomas in Example 1, and competent cells carrying the plasmids were deposited at VKPM, with the following details:
cells introduced with the plasmid containing the insert encoding the amino acid sequence of the heavy chain of b-CTx_R_rec_132 was deposited at VKPM on Apr. 2, 2024 under a deposit number of B-14773; cells introduced with the plasmid containing the insert encoding the amino acid sequence of the light chain of b-CTx_R_rec_132 was deposited at VKPM on Apr. 2, 2024 under a deposit number of B-14772;
cells introduced with the plasmid containing the insert encoding the amino acid sequence of the heavy chain of b-CTx_R_rec_134 was deposited at VKPM on Apr. 2, 2024 under a deposit number of B-14775; cells introduced with the plasmid containing the insert encoding the amino acid sequence of the light chain of b-CTx_R_rec_134 was deposited at VKPM on Apr. 2, 2024 under a deposit number of B-14774;
cells introduced with the plasmid containing the insert encoding the amino acid sequence of the heavy chain of b-CTx_R_rec_137 was deposited at VKPM on Apr. 2, 2024 under a deposit number of B-14777; cells introduced with the plasmid containing the insert encoding the amino acid sequence of the light chain of b-CTx_R_rec_137 was deposited at VKPM on Apr. 2, 2024 under a deposit number of B-14776;
cells introduced with the plasmid containing the insert encoding the amino acid sequence of the heavy chain of b-CTx_R_rec_138 was deposited at VKPM on Apr. 2, 2024 under a deposit number of B-14779; cells introduced with the plasmid containing the insert encoding the amino acid sequence of the light chain of b-CTx_R_rec_138 was deposited at VKPM on Apr. 2, 2024 under a deposit number of B-14778;
cells introduced with the plasmid containing the insert encoding the amino acid sequence of the heavy chain of b-CTx_R_rec_145 was deposited at VKPM on Apr. 2, 2024 under a deposit number of B-14781; cells introduced with the plasmid containing the insert encoding the amino acid sequence of the light chain of b-CTx_R_rec_145 was deposited at VKPM on Apr. 2, 2024 under a deposit number of B-14780;
cells introduced with the plasmid containing the insert encoding the amino acid sequence of the heavy chain of b-CTx_R_rec_150 was deposited at VKPM on Apr. 2, 2024 under a deposit number of B-14783; cells introduced with the plasmid containing the insert encoding the amino acid sequence of the light chain of b-CTx_R_rec_150 was deposited at VKPM on Apr. 2, 2024 under a deposit number of B-14782;
cells introduced with the plasmid containing the insert encoding the amino acid sequence of the heavy chain of b-CTx_R_rec_191 was deposited at VKPM on Apr. 2, 2024 under a deposit number of B-14785; cells introduced with the plasmid containing the insert encoding the amino acid sequence of the light chain of b-CTx_R_rec_191 was deposited at VKPM on Apr. 2, 2024 under a deposit number of B-14784.

Mammalian cell line Expi293F was transfected with molecular gene constructs of recombinant antibodies.

For example, the light chain and heavy chain gene sequences are transferred into the open reading frames (ORF) of expression vectors by homologous recombination or restriction digestion respectively to form the “promoter-antibody light chain gene-terminator” and “promoter-antibody heavy chain gene-terminator” structures on the two expression vectors respectively. Then, by biological, physical or chemical methods, the two expression vectors (antibody light chain expression vector and heavy chain expression vector) were simultaneously transferred into the mammalian cell line Expi293F for expression. Finally, the two light chains and two heavy chains were reassembled into a complete antibody in the cell and secreted into the culture supernatant. Alternatively, the expression vector is modified to contain two open reading frames, and then the light chain and heavy chain gene sequences are inserted into the two open reading frames (ORF) of the expression vector respectively by homologous recombination or restriction digestion to form a “promoter-antibody light chain gene-terminator-vector sequence-promoter-antibody heavy chain gene-terminator” or a “promoter-antibody heavy chain gene-terminator-vector sequence-promoter-antibody light chain gene-terminator” structure. Then, by biological, physical or chemical methods, this expression vector containing both the light and heavy chains of the antibody was transferred into the mammalian cell line Expi293F for expression. Finally, the two light and two heavy chains were reassembled into a complete antibody in the cell and secreted into the culture supernatant.

Antibodies were purified from conditioned media using Protein A affinity chromatography. The chromatography matrix was obtained from GE Healthcare Life Sciences (Piscataway, NJ) and the purification process was performed according to the manufacturer's instructions. Purified monoclonal antibodies were stored in 50% ammonium sulfate as a suspension at 4° C.

### Example 5 Determination of Affinity of Antibodies

The affinity of all anti-β-CTx MAbs were determined by affinity biolayer interferometry (Octet Sartorius, Germany).

Affinity biolayer interferometry was performed according to the following protocol:
Amine Reactive 2nd Generation (AR2G) Octet biosensor was hydrated in deionized water.
The hydrated biosensor was activated with an aqueous solution of 1 mM MEDC (1-ethyl-3-[3-dimethylaminopropyl]carbodiimide hydrochloride) and 0.5 mM s-NHS (N-hydroxysulfosuccinimide) at 25° C. for 5 min.
The synthesized β-CTx peptide EKAH(beta-D)GGR was immobilized on the activated biosensor for 10 min at 25° C.
The biosensor with the β-CTx peptide immobilized was blocked with 1M ethanolamine (pH 8.5) at 25° C. for 5 min.

The antibody solutions (1, 2, 5 g/ml) in PBS were applied to the sensor according to the following protocol:
hydrating the sensor;
binding the antibody to the sensor;
cleaning the sensor;
blocking the sensor;
association (perform association for each concentration of antibody separately);
dissociation;
sensor regeneration;

The affinity constants of antibodies are shown in Table 5 below.

TABLE 5

| Name of MAb | KD (M) |
|---|---|
| b-CTx_M_hyb_015 | 5.3E−9 |
| b-CTx_M_hyb_061 | 4.1E−9 |
| b-CTx_M_hyb_063 | 3.4E−9 |
| b-CTx_R_rec_132 | 8.5E−10 |
| b-CTx_R_rec_134 | 1.1E−9 |
| b-CTx_R_rec_137 | 2.7E−9 |
| b-CTx_R_rec_138 | 2.0E−9 |
| b-CTx_R_rec_145 | 8.9E−10 |
| b-CTx_R_rec_150 | 1.1E−9 |
| b-CTx_R_rec_191 | 7.1E−9 |

It can be seen from Table 5 that all antibodies have good ability to bind to β-CTx, the KD of b-CTx_R_rec_145 reaches 8.9E-10, and the KD of b-CTx_R_rec_132 reaches 8.5E-10.

### Example 6 Development of Chemiluminescent Sandwich Immunoassay

Different paired combinations of all MAbs were tested using a sandwich immunoassay to find the combination with the best characteristics.

For chemiluminescent sandwich particle-based immunoassay, detection MAbs were labeled with alkaline phosphatase (cat #03137031103, Roche Custom Biotech) and capture MAbs were labeled with biotin (Cat #PG82075, Thermo). The capture antibodies were incubated with streptavidin-labeled particles (MyOne T1 Dynabeads Streptavidin PMP Hydrophobic particles 100 mkm, Cat #35604D, Thermo), and the antibody-labeled particles were used for detection in a solution of 0.25 mg/ml 50 mM Tris-HCl, pH 7.4, 150 mM NaCl, 2% BSA, 0.2% Tween-20, 0.05% ProClin 300, 0.09% NaN₃. The detection antibodies were used at a concentration of 0.5 g/ml in a solution of 50 mM MES, pH6, 0.9% NaCl, 1% BSA, 0.05% Tween-20, 0.048% ProClin 300, 2 mM MgCl₂, 0.1 mM ZnCl₂. The antibody pairs used (capture-detection) are listed in Table 6.

Pooled human plasma samples with pre-measured β-CTx concentrations were employed as calibrators in the immunoassay. Concentrations of β-CTx in calibrator plasma samples were determined using the Roche beta-CrossLaps immunoassay on a Cobas 6000 automated analyzer (Roche Diagnostics GmbH). Pooled human plasma samples were serially diluted in a solution of 50 mM MES, pH 6, 0.9% NaCl, 1% BSA, 0.05% Tween-20, 0.048% ProClin 300, 2 mM MgCl₂, 0.1 mM ZnCl₂. 50 μL of calibrator plasma was mixed with 50 μL of particle suspension and 50 μL of detection MAb-phosphatase conjugate solution. A one-step immunoassay was performed in an automated analyzer Mindray CL-6000i according to the manufacturer's instructions. Chemiluminescence was detected using the substrate solution provided together with the analyzer. Chemiluminescence is expressed in relative light units (RLU).

Serum supplemented with 150 ng/mL α-CTx was used to test the α-CTx cross-reactivity of different antibody pairs. The cross-reactivity percentage was calculated from the measured concentration/supplemented concentration.

In addition, the limit of detection (LOD) for different antibody pairs was tested based on the following protocol: Limit of Blank (LOB) and LOD were determined according to suggestions of Clinical and Laboratory Standards Institute (CLSI) (Evaluation of Detection Capability for Clinical Laboratory Measurement Procedures; Approved Guideline—Second Edition; EP 17-A Vol. 24 No. 34).

Twenty replicates of zero analytes (50 mM MES, pH 6, 0.90% NaCl, 1 BSA, 0.05% Tween-20, 0.0485 ProClin 300, 2 mM MgCl₂, 0.1 mM ZnCl₂) were measured using the corresponding chemiluminescent sandwich immunoassay.

The calculation formulas are

$$LOB = 1.64 * Mean ;$$

$$LOD = LOB + 1.65 * SD$$

Test results of cross-reactivity and LOD are listed in Table 6.

TABLE 6

| | | LOD | Cross-reactivity |
|---|---|---|---|
| Capture MAb | Detection MAb | pg/ml | with α-CTx |
| b-CTx_M_hyb_061 | b-CTx_R_rec_145 | 4.0 | 0.02% |
| b-CTx_M_hyb_063 | b-CTx_R_rec_145 | 4.1 | 0.01% |
| b-CTx_R_rec_145 | b-CTx_R_rec_138 | 3.3 | 0.02% |
| b-CTx_M_hyb_015 | b-CTx_R_rec_137 | 10.9 | 0.20% |
| b-CTx_M_hyb_015 | b-CTx_R_rec_191 | 10.0 | 0.14% |
| b-CTx_M_hyb_015 | b-CTx_R_rec_145 | 9.7 | 0.14% |
| b-CTx_M_hyb_061 | b-CTx_R_rec_132 | 7.0 | 0.03% |
| b-CTx_R_rec_132 | b-CTx_R_rec_138 | 6.4 | 0.01% |
| b-CTx_R_rec_132 | b-CTx_R_rec_145 | 6.1 | 0.02% |
| b-CTx_R_rec_134 | b-CTx_R_rec_138 | 6.6 | 0.03% |
| b-CTx_R_rec_134 | b-CTx_R_rec_145 | 6.3 | 0.00% |
| b-CTx_R_rec_137 | b-CTx_R_rec_138 | 5.3 | 0.02% |
| b-CTx_R_rec_137 | b-CTx_R_rec_145 | 4.6 | 0.03% |
| b-CTx_R_rec_191 | b-CTx_R_rec_138 | 6.0 | 0.06% |
| b-CTx_R_rec_191 | b-CTx_R_rec_145 | 5.8 | 0.01% |
| b-CTx_R_rec_150 | b-CTx_R_rec_132 | 7.2 | 0.01% |
| b-CTx_R_rec_150 | b-CTx_R_rec_137 | 7.1 | 0.00% |
| b-CTx_R_rec_191 | b-CTx_R_rec_132 | 6.2 | 0.03% |
| b-CTx_R_rec_191 | b-CTx_M_hyb_061 | 7.0 | 0.17% |
| b-CTx_M_hyb_061 | b-CTx_R_rec_134 | 7.1 | 0.03% |
| b-CTx_M_hyb_061 | b-CTx_R_rec_137 | 7.0 | 0.02% |
| b-CTx_M_hyb_063 | b-CTx_R_rec_137 | 7.3 | 0.06% |

It can be seen from Table 6 that the cross-reactivity percentage with ax-CTx for all tested antibody pairs was 0.20% or below, and for some antibody pairs was even 0.05% or below. The LOD of all antibody pairs tested was 10.9 μg/ml or below, and for some antibody pairs was even as low as 5 μg/ml. The LOB and LOD of sandwich immunoassay using the combination of b-CTx_R_rec_145 and b-CTx_R_rec_138 were 2.0 and 3.3 μg/ml, respectively.

### Example 7 Antibody Epitope Mapping

As shown in Table 7, the precise epitopes of all anti-β-CTx MAbs were determined by using extended or truncated peptides.

Enzyme-linked immunosorbent assay (ELISA) using β-CTx peptide analogues conjugated with ovalbumin as pre-adsorbed antigen was adopted for detecting β-CTx-specific antibodies. Each well was added with 2020 ng/0.05 ml β-CTx peptide analogue-ovalbumin and incubated under RT conditions with gentle agitation. After 30 minutes of antigen adsorption, the plates were washed twice with PBS containing 0.1% detergent Tween 20 (PBST). The plates were then incubated with 0.05 ml of 1 g/ml antibodies for 30 min and washed twice with PBST. HRP-labeled anti-mouse IgG or anti-rabbit IgG secondary polyclonal antibodies (0.05 ml per well, diluted with PBST at 1:1000) was added and incubated for 30 min to detect mouse or rabbit antibodies bound to pre-adsorbed antigen. The secondary antibodies were obtained from Sigma Chemicals, St. Louise, Mo. After incubation with the secondary antibody, the plates were washed six times with PBST, and then 0.05 ml of 3,3′,5,5′-tetramethylbenzidine (TMB) peroxidase substrate solution containing 0.03% hydrogen peroxide was added. After incubation for 10 min, 0.05 ml of 1M phosphoric acid was added to stop the reaction, and the absorbance in the wells was measured at a wavelength of 450 nm. Absorbance was measured using a Labsystems Multiscan microplate reader (Labsystems, Finland).

TABLE 7

| | Ova- | EKAH- | Ova- | Ova- | Ova- | Ova- | | |
|---|---|---|---|---|---|---|---|---|
| | CEKAH- | (betaD)- | CEKAH- | CGGAH- | CEKAH- | CEKAH- | | |
| | (betaD)- | GGRG | (betaD)- | (betaD)- | (betaD)- | (betaD) | Background | |
| Antibody | GGR | GC-Ova | GGRYYRA | GGR | GG | G | value | Epitope |
| b-CTx_M_hyb_015 | 1.658 | 1.510 | 1.497 | 1.453 | 1.048 | 0.899 | 0.154 | AH(beta |
| | | | | | | | | D)G |
| b-CTx_M_hyb_061 | 1.647 | 1.273 | 1.220 | 2.013 | 0.061 | 0.049 | 0.045 | AH(beta |
| | | | | | | | | D)GGR |
| b-CTx_M_hyb_063 | 1.884 | 1.675 | 1.677 | 1.806 | 0.058 | 0.059 | 0.044 | AH(beta |
| | | | | | | | | D)GGR |
| b-CTx_R_rec_132 | 1.400 | 0.412 | 0.467 | 1.299 | 0.139 | 0.121 | 0.045 | AH(beta |
| | | | | | | | | D)G |
| b-CTx_R_rec_134 | 1.227 | 0.361 | 0.302 | 1.292 | 0.141 | 0.089 | 0.044 | AH(beta |
| | | | | | | | | D)GG/ |
| | | | | | | | | AH(beta |
| | | | | | | | | D)G |
| b-CTx_R_rec_137 | 1.266 | 0.422 | 0.326 | 1.315 | 0.099 | 0.069 | 0.044 | AH(beta |
| | | | | | | | | D)GG |
| b-CTx_R_rec_138 | 1.293 | 0.413 | 0.767 | 1.807 | 0.070 | 0.062 | 0.042 | AH(beta |
| | | | | | | | | D)GGR |
| b-CTx_R_rec_145 | 1.335 | 0.812 | 0.733 | 1.519 | 0.521 | 0.185 | 0.044 | AH(beta |
| | | | | | | | | D)G |
| b-CTx_R_rec_150 | 1.436 | 0.063 | 0.142 | 1.364 | 0.082 | 0.079 | 0.046 | AH(beta |
| | | | | | | | | D)GGR- |
| | | | | | | | | COOH |
| b-CTx_R_rec_191 | 0.975 | 0.045 | 0.089 | 1.738 | 0.073 | 0.079 | 0.043 | AH(beta |
| | | | | | | | | D)GGR- |
| | | | | | | | | COOH |

It can be seen from Table 7 that the epitopes of all anti-β-CTx antibodies have 4-6 amino acid residues in the β-CTx octapeptide. Antibody b-CTx_R_rec_134 binds to both epitopes AH(betaD)GG and AH(betaD)G. Antibodies b-CTx_R_rec_150 and b-CTx_R_rec_191 effectively recognize β-CTx in the presence of a C-terminal arginine residue with a free carboxyl group.

### Example 8 Assessment on Sample Stability

### 8.1 Preparation of Kits

### Step 1: Preparation of Reagent R1

Streptavidin and superparamagnetic particles were mixed in TBS buffer, and after sufficient reaction, the mixture was placed on a magnetic separator until the supernatant became clear. Then, the supernatant was removed, and the streptavidin-coated superparamagnetic particles were retained and washed three times with TBS buffer, and finally dissolved with R1 diluent to achieve the final superparamagnetic particle content of 0.05%, thus obtaining the reagent R1, which was refrigerated at 2˜8° C. The components of reagent R1 may be prepared by conventional methods.

### Step 2: Preparation of Reagent R2

A detection antibody was conjugated to alkaline phosphatase, wherein the molar ratio of the conjugate to the detection antibody was 15:1. Then, the conjugate was dissolved with R2 diluent to achieve the final concentration of 2 μg/mL, thus obtaining reagent R2, which was refrigerated at 2˜8° C. The components of reagent R2 may be prepared by conventional methods.

### Step 3: Preparation of Reagent R3

A capture antibody was conjugated to biotin, wherein the molar ratio of the conjugate to the capture antibody was 30:1. Then, the conjugate was dissolved with R3 diluent to achieve the final concentration of 1 μg/mL, thus obtaining reagent R3, which was refrigerated at 2˜8° C. The components of reagent R3 may be prepared by conventional methods.

### Step 4: Preparation of β-CTx Calibrators

β-CTx calibrators were prepared by diluting β-CTx antigen to concentration points of 0 ng/mL, 0.07 ng/mL, 0.18 ng/mL, 0.32 ng/mL, 0.48 ng/mL, 0.82 ng/mL, 1.34 ng/mL, 2.81 ng/mL, 3.86 ng/mL, 5.62 ng/mL, and 6.57 ng/mL.

Kits were prepared separately using the antibody pairs listed in Table 8 below for subsequent experiments.

TABLE 8

| Kit | Capture antibody | Detection antibody |
|---|---|---|
| A | b-CTx_R_rec_132 | b-CTx_R_rec_191 |
| B | b-CTx_M_hyb_061 | b-CTx_R_rec_150 |
| C | b-CTx_R_rec_145 | b-CTx_R_rec_138 |
| D | b-CTx_M_hyb_063 | b-CTx_R_rec_138 |
| E | b-CTx_R_rec_134 | b-CTx_R_rec_145 |
| F | b-CTx_M_hyb_015 | b-CTx_R_rec_137 |

### 8.2 Measurement Procedures of the β-CTx Kit:

(1) The β-CTx kit (including reagent R1, reagent R2 and reagent R3) was loaded into the Mindray CL series automatic chemiluminescent immunoanalyzer.
(2) The sample volume required by the system for each measurement was 50 μL, and the total measurement time per report was 32 min.
(3) The specific sample loading process of the detection system was as follows: 50 μL of sample and 50 μL of reagent R3 was added to a reaction tube. After incubation, the β-CTx in the sample bound to the anti-β-CTx antibody in R3. Then, 50 μL of reagent R1 and 50 μL of reagent R2 were added at the same time. After incubation, the labeled anti-β-CTx antibody bound to another site of β-CTx in the sample to form a sandwich complex. After the reaction was completed, the magnetic beads were immobilized using a magnetic field and unbound substances were removed through washing.

The reaction substrate or excitation liquid was then added to the reaction tube to generate an optical signal, and the number of photons generated was measured. The number of photons generated was in direct proportion to the concentration T R-CTx in the sample.
(4) The matched β-CTx calibrator was used for calibration test. Based on the calibration data, the system software mathematically fitted the optical signal to the concentration by using a weighted four-parameter logarithmic curve (4PLC), and the final result was given in a concentration form of ng/mL.
8.3 The kits prepared using b-CTx_R_rec_132 and b-CTx_R_rec_191 were used to detect different samples according to the measurement procedures of 8.2. The results are shown in Table 9.

TABLE 9

| | | | |
|---|---|---|---|
| | 0 h | 12 h | |
| b-CTx_R_rec_132 and | concentration- | concentration- | |
| b-CTx_R_rec_191 | ng/mL | ng/mL | Variation |

Serum-sample stability (20~25° C.)

| | | | |
|---|---|---|---|
| Serum sample-1 | 0.637 | 0.608 | −4.6% |
| Serum sample-2 | 0.227 | 0.217 | −4.3% |
| Serum sample-3 | 0.392 | 0.385 | −1.8% |
| Serum sample-4 | 0.129 | 0.121 | −5.6% |
| Serum sample-5 | 0.672 | 0.620 | −7.9% |
| Serum sample-6 | 0.186 | 0.175 | −5.8% |
| Serum sample-7 | 0.475 | 0.453 | −4.7% |
| Serum sample-8 | 0.448 | 0.426 | −4.9% |
| Serum sample-9 | 0.368 | 0.352 | −4.2% |
| Serum sample-10 | 0.339 | 0.325 | −4.0% |
| | | Mean | −4.8% |
| | | variation | |

Serum-sample stability (2~8° C.)

| | | | |
|---|---|---|---|
| Serum sample-11 | 0.613 | 0.634 | 3.3% |
| Serum sample-12 | 0.479 | 0.487 | 1.5% |
| Serum sample-13 | 0.913 | 0.834 | −8.7% |
| Serum sample-14 | 0.324 | 0.332 | 2.4% |
| Serum sample-15 | 0.648 | 0.621 | −4.2% |
| Serum sample-16 | 0.148 | 0.149 | 0.7% |
| Serum sample-17 | 0.353 | 0.326 | −7.6% |
| Serum sample-18 | 0.215 | 0.208 | −3.3% |
| Serum sample-19 | 0.530 | 0.522 | −1.6% |
| Serum sample-20 | 0.384 | 0.375 | −2.5% |
| | | Mean | −2.0% |
| | | variation | |

Heparin lithium-sample stability (20~25° C.)

| | | | |
|---|---|---|---|
| Heparin lithium | 0.660 | 0.674 | 2.1% |
| sample-1 | | | |
| Heparin lithium | 0.249 | 0.258 | 3.5% |
| sample-2 | | | |
| Heparin lithium | 0.423 | 0.398 | −5.9% |
| sample-3 | | | |
| Heparin lithium | 0.136 | 0.127 | −6.5% |
| sample-4 | | | |
| Heparin lithium | 0.735 | 0.679 | −7.7% |
| sample-5 | | | |
| Heparin lithium | 0.182 | 0.170 | −6.8% |
| sample-6 | | | |
| Heparin lithium | 0.493 | 0.477 | −3.1% |
| sample-7 | | | |
| Heparin lithium | 0.477 | 0.443 | −7.1% |
| sample-8 | | | |
| Heparin lithium | 0.389 | 0.359 | −7.7% |
| sample-9 | | | |
| Heparin lithium | 0.362 | 0.337 | −6.8% |
| sample-10 | | | |
| | | Mean | −4.6% |
| | | variation | |

Heparin lithium-sample stability (2~8° C.)

| | | | |
|---|---|---|---|
| Heparin lithium | 0.690 | 0.655 | −5.0% |
| sample-11 | | | |
| Heparin lithium | 0.476 | 0.486 | 2.0% |
| sample-12 | | | |
| Heparin lithium | 0.944 | 0.864 | −8.4% |
| sample-13 | | | |
| Heparin lithium | 0.351 | 0.380 | 8.1% |
| sample-14 | | | |
| Heparin lithium | 0.702 | 0.663 | −5.5% |
| sample-15 | | | |
| Heparin lithium | 0.175 | 0.187 | 6.7% |
| sample-16 | | | |
| Heparin lithium | 0.391 | 0.386 | −1.1% |
| sample-17 | | | |
| Heparin lithium | 0.208 | 0.221 | 6.6% |
| sample-18 | | | |
| Heparin lithium | 0.571 | 0.561 | −1.8% |
| sample-19 | | | |
| Heparin lithium | 0.413 | 0.422 | 2.2% |
| sample-20 | | | |
| | | Mean | 0.4% |
| | | variation | |

EDTA-K2-sample stability (20~25° C.)

| | | | |
|---|---|---|---|
| EDTA-K2 sample-1 | 0.683 | 0.652 | −4.6% |
| EDTA-K2 sample-2 | 0.247 | 0.269 | 8.9% |
| EDTA-K2 sample-3 | 0.406 | 0.376 | −7.6% |
| EDTA-K2 sample-4 | 0.135 | 0.136 | 1.2% |
| EDTA-K2 sample-5 | 0.653 | 0.645 | −1.2% |
| EDTA-K2 sample-6 | 0.163 | 0.163 | −0.3% |
| EDTA-K2 sample-7 | 0.421 | 0.390 | −7.4% |
| EDTA-K2 sample-8 | 0.425 | 0.409 | −3.7% |
| EDTA-K2 sample-9 | 0.332 | 0.329 | −0.9% |
| EDTA-K2 sample-10 | 0.290 | 0.301 | 3.8% |
| | | Mean | −1.2% |
| | | variation | |

EDTA-K2-sample stability (2~8° C.)

| | | | |
|---|---|---|---|
| EDTA-K2 sample-11 | 0.635 | 0.657 | 3.6% |
| EDTA-K2 sample-12 | 0.434 | 0.429 | −1.1% |
| EDTA-K2 sample-13 | 0.978 | 0.993 | 1.5% |
| EDTA-K2 sample-14 | 0.314 | 0.316 | 0.6% |
| EDTA-K2 sample-15 | 0.624 | 0.639 | 2.5% |
| EDTA-K2 sample-16 | 0.162 | 0.169 | 4.3% |
| EDTA-K2 sample-17 | 0.319 | 0.336 | 5.2% |
| EDTA-K2 sample-18 | 0.194 | 0.198 | 2.1% |
| EDTA-K2 sample-19 | 0.514 | 0.559 | 8.8% |
| EDTA-K2 sample-20 | 0.387 | 0.393 | 1.6% |
| | | Mean | 2.9% |
| | | variation | |

8.4 The kits prepared using b-CTxM_hyb_061 and b-CTx_R_rec_150 were used to detect different samples according to the measurement procedures of 8.2. The results are shown in Table 10.

TABLE 10

| | | | |
|---|---|---|---|
| | 0 h | 12 h | |
| b-CTx_M_hyb_061 and | concentration- | concentration- | |
| b-CTx_R_rec_150 | ng/mL | ng/mL | Variation |

Serum-sample stability (20~25° C.)

| | | | |
|---|---|---|---|
| Serum sample-1 | 0.658 | 0.715 | 8.7% |
| Serum sample-2 | 0.217 | 0.205 | −5.9% |
| Serum sample-3 | 0.406 | 0.398 | −2.0% |
| Serum sample-4 | 0.132 | 0.139 | 5.5% |
| Serum sample-5 | 0.734 | 0.689 | −6.2% |
| Serum sample-6 | 0.193 | 0.180 | −6.9% |
| Serum sample-7 | 0.523 | 0.484 | −7.5% |
| Serum sample-8 | 0.434 | 0.468 | 7.7% |
| Serum sample-9 | 0.361 | 0.338 | −6.4% |
| Serum sample-10 | 0.362 | 0.337 | −6.9% |
| | | Mean | −2.0% |
| | | variation | |

Serum-sample stability (2~8° C.)

| | | | |
|---|---|---|---|
| Serum sample-11 | 0.653 | 0.657 | 0.5% |
| Serum sample-12 | 0.545 | 0.512 | −6.0% |
| Serum sample-13 | 0.910 | 0.852 | −6.4% |
| Serum sample-14 | 0.328 | 0.321 | −2.0% |
| Serum sample-15 | 0.730 | 0.707 | −3.2% |
| Serum sample-16 | 0.151 | 0.161 | 6.9% |
| Serum sample-17 | 0.453 | 0.425 | −6.3% |
| Serum sample-18 | 0.251 | 0.230 | −8.3% |
| Serum sample-19 | 0.452 | 0.412 | −8.8% |
| Serum sample-20 | 0.428 | 0.396 | −7.6% |
| | | Mean | −4.1% |
| | | variation | |

Heparin lithium-sample stability (20~25° C.)

| | | | |
|---|---|---|---|
| Heparin lithium | 0.765 | 0.728 | −4.8% |
| sample-1 | | | |
| Heparin lithium | 0.244 | 0.234 | −4.4% |
| sample-2 | | | |
| Heparin lithium | 0.416 | 0.391 | −6.0% |
| sample-3 | | | |
| Heparin lithium | 0.129 | 0.128 | −0.7% |
| sample-4 | | | |
| Heparin lithium | 0.756 | 0.689 | −8.8% |
| sample-5 | | | |
| Heparin lithium | 0.182 | 0.172 | −5.6% |
| sample-6 | | | |
| Heparin lithium | 0.555 | 0.509 | −8.4% |
| sample-7 | | | |
| Heparin lithium | 0.473 | 0.440 | −7.0% |
| sample-8 | | | |
| Heparin lithium | 0.345 | 0.358 | 3.6% |
| sample-9 | | | |
| Heparin lithium | 0.408 | 0.374 | −8.4% |
| sample-10 | | | |
| | | Mean | −5.0% |
| | | variation | |

Heparin lithium-sample stability (2~8° C.)

| | | | |
|---|---|---|---|
| Heparin lithium | 0.678 | 0.620 | −8.5% |
| sample-11 | | | |
| Heparin lithium | 0.488 | 0.482 | −1.3% |
| sample-12 | | | |
| Heparin lithium | 0.950 | 0.892 | −6.2% |
| sample-13 | | | |
| Heparin lithium | 0.359 | 0.381 | 6.3% |
| sample-14 | | | |
| Heparin lithium | 0.721 | 0.690 | −4.2% |
| sample-15 | | | |
| Heparin lithium | 0.166 | 0.155 | −6.9% |
| sample-16 | | | |
| Heparin lithium | 0.395 | 0.368 | −6.8% |
| sample-17 | | | |
| Heparin lithium | 0.209 | 0.190 | −9.0% |
| sample-18 | | | |
| Heparin lithium | 0.564 | 0.573 | 1.6% |
| sample-19 | | | |
| Heparin lithium | 0.377 | 0.365 | −3.2% |
| sample-20 | | | |
| | | Mean | −3.8% |
| | | variation | |

EDTA-K2-sample stability (20~25° C.)

| | | | |
|---|---|---|---|
| EDTA-K2 sample-1 | 0.697 | 0.669 | −4.0% |
| EDTA-K2 sample-2 | 0.268 | 0.265 | −0.9% |
| EDTA-K2 sample-3 | 0.400 | 0.390 | −2.5% |
| EDTA-K2 sample-4 | 0.131 | 0.140 | 7.1% |
| EDTA-K2 sample-5 | 0.700 | 0.651 | −7.1% |
| EDTA-K2 sample-6 | 0.179 | 0.182 | 1.7% |
| EDTA-K2 sample-7 | 0.512 | 0.494 | −3.5% |
| EDTA-K2 sample-8 | 0.431 | 0.413 | −4.1% |
| EDTA-K2 sample-9 | 0.352 | 0.324 | −8.1% |
| EDTA-K2 sample-10 | 0.316 | 0.300 | −5.1% |
| | | Mean | −2.6% |
| | | variation | |

EDTA-K2-sample stability (2~8° C.)

| | | | |
|---|---|---|---|
| EDTA-K2 sample-11 | 0.665 | 0.667 | 0.4% |
| EDTA-K2 sample-12 | 0.423 | 0.410 | −3.2% |
| EDTA-K2 sample-13 | 0.983 | 0.900 | −8.4% |
| EDTA-K2 sample-14 | 0.311 | 0.320 | 2.8% |
| EDTA-K2 sample-15 | 0.697 | 0.704 | 0.9% |
| EDTA-K2 sample-16 | 0.157 | 0.144 | −8.4% |
| EDTA-K2 sample-17 | 0.404 | 0.377 | −6.7% |
| EDTA-K2 sample-18 | 0.190 | 0.194 | 2.2% |
| EDTA-K2 sample-19 | 0.512 | 0.535 | 4.6% |
| EDTA-K2 sample-20 | 0.447 | 0.413 | −7.6% |
| | | Mean | −2.3% |
| | | variation | |

8.5 The kits prepared using b-CTx_R_rec_145 and b-CTx_R_rec_138 were used to detect different samples according to the measurement procedures of 8.2. The results are shown in Table 11.

TABLE 11

| | | | |
|---|---|---|---|
| | 0 h | 12 h | |
| b-CTx_R_rec_145 and | concentration- | concentration- | |
| b-CTx_R_rec_138 | ng/mL | ng/mL | Variation |

Serum-sample stability (20~25° C.)

| | | | |
|---|---|---|---|
| Serum sample-1 | 0.684 | 0.623 | −8.9% |
| Serum sample-2 | 0.238 | 0.220 | −7.4% |
| Serum sample-3 | 0.389 | 0.394 | 1.4% |
| Serum sample-4 | 0.124 | 0.126 | 1.8% |
| Serum sample-5 | 0.643 | 0.604 | −6.1% |
| Serum sample-6 | 0.180 | 0.169 | −5.8% |
| Serum sample-7 | 0.468 | 0.472 | 0.7% |
| Serum sample-8 | 0.469 | 0.472 | 0.8% |
| Serum sample-9 | 0.348 | 0.344 | −1.1% |
| Serum sample-10 | 0.317 | 0.322 | 1.3% |
| | | Mean | −2.3% |
| | | variation | |

Serum-sample stability (2~8° C.)

| | | | |
|---|---|---|---|
| Serum sample-11 | 0.596 | 0.607 | 1.8% |
| Serum sample-12 | 0.466 | 0.482 | 3.4% |
| Serum sample-13 | 0.928 | 0.860 | −7.4% |
| Serum sample-14 | 0.323 | 0.328 | 1.7% |
| Serum sample-15 | 0.629 | 0.653 | 3.7% |
| Serum sample-16 | 0.159 | 0.148 | −6.8% |
| Serum sample-17 | 0.349 | 0.328 | −5.9% |
| Serum sample-18 | 0.214 | 0.204 | −4.6% |
| Serum sample-19 | 0.484 | 0.461 | −4.9% |
| Serum sample-20 | 0.384 | 0.372 | −3.1% |
| | | Mean | −2.2% |
| | | variation | |

Heparin lithium-sample stability (20~25° C.)

| | | | |
|---|---|---|---|
| Heparin lithium | 0.702 | 0.675 | −3.8% |
| sample-1 | | | |
| Heparin lithium | 0.268 | 0.251 | −6.1% |
| sample-2 | | | |
| Heparin lithium | 0.417 | 0.388 | −6.9% |
| sample-3 | | | |
| Heparin lithium | 0.135 | 0.124 | −8.2% |
| sample-4 | | | |
| Heparin lithium | 0.735 | 0.663 | −9.7% |
| sample-5 | | | |
| Heparin lithium | 0.175 | 0.163 | −7.1% |
| sample-6 | | | |
| Heparin lithium | 0.491 | 0.478 | −2.8% |
| sample-7 | | | |
| Heparin lithium | 0.501 | 0.453 | −9.5% |
| sample-8 | | | |
| Heparin lithium | 0.410 | 0.377 | −8.2% |
| sample-9 | | | |
| Heparin lithium | 0.349 | 0.330 | −5.6% |
| sample-10 | | | |
| | | Mean | −6.8% |
| | | variation | |

Heparin lithium-sample stability (2~8° C.)

| | | | |
|---|---|---|---|
| Heparin lithium | 0.677 | 0.647 | −4.5% |
| sample-11 | | | |
| Heparin lithium | 0.457 | 0.471 | 3.1% |
| sample-12 | | | |
| Heparin lithium | 0.953 | 0.884 | −7.2% |
| sample-13 | | | |
| Heparin lithium | 0.343 | 0.313 | −8.9% |
| sample-14 | | | |
| Heparin lithium | 0.687 | 0.651 | −5.3% |
| sample-15 | | | |
| Heparin lithium | 0.173 | 0.184 | 6.4% |
| sample-16 | | | |
| Heparin lithium | 0.374 | 0.344 | −8.1% |
| sample-17 | | | |
| Heparin lithium | 0.205 | 0.190 | −7.3% |
| sample-18 | | | |
| Heparin lithium | 0.567 | 0.554 | −2.2% |
| sample-19 | | | |
| Heparin lithium | 0.412 | 0.405 | −1.7% |
| sample-20 | | | |
| | | Mean | −3.6% |
| | | variation | |

EDTA-K2-sample stability (20~25° C.)

| | | | |
|---|---|---|---|
| EDTA-K2 sample-1 | 0.680 | 0.642 | −5.6% |
| EDTA-K2 sample-2 | 0.243 | 0.261 | 7.2% |
| EDTA-K2 sample-3 | 0.421 | 0.383 | −8.9% |
| EDTA-K2 sample-4 | 0.135 | 0.135 | 0.2% |
| EDTA-K2 sample-5 | 0.652 | 0.625 | −4.2% |
| EDTA-K2 sample-6 | 0.162 | 0.161 | −0.8% |
| EDTA-K2 sample-7 | 0.425 | 0.385 | −9.4% |
| EDTA-K2 sample-8 | 0.423 | 0.386 | −8.7% |
| EDTA-K2 sample-9 | 0.328 | 0.320 | −2.4% |
| EDTA-K2 sample-10 | 0.285 | 0.294 | 3.1% |
| | | Mean | −2.9% |
| | | variation | |

EDTA-K2-sample stability (2~8° C.)

| | | | |
|---|---|---|---|
| EDTA-K2 sample-11 | 0.657 | 0.660 | 0.4% |
| EDTA-K2 sample-12 | 0.442 | 0.422 | −4.6% |
| EDTA-K2 sample-13 | 0.995 | 0.953 | −4.2% |
| EDTA-K2 sample-14 | 0.323 | 0.318 | −1.4% |
| EDTA-K2 sample-15 | 0.659 | 0.618 | −6.3% |
| EDTA-K2 sample-16 | 0.166 | 0.159 | −4.4% |
| EDTA-K2 sample-17 | 0.329 | 0.303 | −7.9% |
| EDTA-K2 sample-18 | 0.204 | 0.206 | 1.2% |
| EDTA-K2 sample-19 | 0.538 | 0.501 | −6.9% |
| EDTA-K2 sample-20 | 0.402 | 0.387 | −3.8% |
| | | Mean | −3.8% |
| | | variation | |

8.6 The kits prepared using b-CTxM_hyb_063 and b-CTx_R_rec_138 were used to detect different samples according to the measurement procedures of 8.2. The results are shown in Table 12.

TABLE 12

| | | | |
|---|---|---|---|
| | 0 h | 12 h | |
| b-CTx_M_hyb_063 and | concentration- | concentration- | |
| b-CTx_R_rec_138 | ng/mL | ng/mL | Variation |

Serum-sample stability (20~25° C.)

| | | | |
|---|---|---|---|
| Serum sample-1 | 0.647 | 0.603 | −6.7% |
| Serum sample-2 | 0.234 | 0.218 | −6.9% |
| Serum sample-3 | 0.385 | 0.377 | −1.9% |
| Serum sample-4 | 0.129 | 0.123 | −4.8% |
| Serum sample-5 | 0.675 | 0.622 | −7.9% |
| Serum sample-6 | 0.191 | 0.175 | −8.1% |
| Serum sample-7 | 0.483 | 0.467 | −3.4% |
| Serum sample-8 | 0.459 | 0.473 | 3.1% |
| Serum sample-9 | 0.368 | 0.350 | −5.0% |
| Serum sample-10 | 0.353 | 0.330 | −6.7% |
| | | Mean | −4.8% |
| | | variation | |

Serum-sample stability (2~8° C.)

| | | | |
|---|---|---|---|
| Serum sample-1 | 0.620 | 0.608 | −1.9% |
| Serum sample-2 | 0.480 | 0.477 | −0.7% |
| Serum sample-3 | 0.921 | 0.851 | −7.7% |
| Serum sample-4 | 0.340 | 0.326 | −3.9% |
| Serum sample-5 | 0.634 | 0.645 | 1.8% |
| Serum sample-6 | 0.148 | 0.141 | −4.7% |
| Serum sample-7 | 0.356 | 0.356 | 0.1% |
| Serum sample-8 | 0.219 | 0.200 | −8.8% |
| Serum sample-9 | 0.505 | 0.497 | −1.6% |
| Serum sample-10 | 0.393 | 0.378 | −3.8% |
| | | Mean | −3.1% |
| | | variation | |

Heparin lithium-sample stability (20~25° C.)

| | | | |
|---|---|---|---|
| Heparin lithium | 0.743 | 0.674 | −9.3% |
| sample-1 | | | |
| Heparin lithium | 0.274 | 0.254 | −7.2% |
| sample-2 | | | |
| Heparin lithium | 0.421 | 0.380 | −9.7% |
| sample-3 | | | |
| Heparin lithium | 0.137 | 0.128 | −6.3% |
| sample-4 | | | |
| Heparin lithium | 0.681 | 0.664 | −2.4% |
| sample-5 | | | |
| Heparin lithium | 0.182 | 0.166 | −8.8% |
| sample-6 | | | |
| Heparin lithium | 0.511 | 0.471 | −7.7% |
| sample-7 | | | |
| Heparin lithium | 0.487 | 0.460 | −5.6% |
| sample-8 | | | |
| Heparin lithium | 0.394 | 0.357 | −9.2% |
| sample-9 | | | |
| Heparin lithium | 0.360 | 0.369 | 2.6% |
| sample-10 | | | |
| | | Mean | −6.4% |
| | | variation | |

Heparin lithium-sample stability (2~8° C.)

| | | | |
|---|---|---|---|
| Heparin lithium | 0.686 | 0.646 | −5.9% |
| sample-11 | | | |
| Heparin lithium | 0.492 | 0.496 | 0.9% |
| sample-12 | | | |
| Heparin lithium | 0.976 | 0.891 | −8.8% |
| sample-13 | | | |
| Heparin lithium | 0.349 | 0.328 | −6.2% |
| sample-14 | | | |
| Heparin lithium | 0.659 | 0.649 | −1.5% |
| sample-15 | | | |
| Heparin lithium | 0.175 | 0.162 | −7.3% |
| sample-16 | | | |
| Heparin lithium | 0.376 | 0.387 | 3.0% |
| sample-17 | | | |
| Heparin lithium | 0.209 | 0.197 | −5.7% |
| sample-18 | | | |
| Heparin lithium | 0.537 | 0.522 | −2.9% |
| sample-19 | | | |
| Heparin lithium | 0.437 | 0.406 | −6.9% |
| sample-20 | | | |
| | | Mean | −4.1% |
| | | variation | |

EDTA-K2-sample stability (20~25° C.)

| | | | |
|---|---|---|---|
| EDTA-K2 sample-1 | 0.686 | 0.633 | −7.7% |
| EDTA-K2 sample-2 | 0.251 | 0.269 | 7.0% |
| EDTA-K2 sample-3 | 0.420 | 0.383 | −8.8% |
| EDTA-K2 sample-4 | 0.130 | 0.136 | 4.4% |
| EDTA-K2 sample-5 | 0.640 | 0.641 | 0.2% |
| EDTA-K2 sample-6 | 0.160 | 0.162 | 0.8% |
| EDTA-K2 sample-7 | 0.415 | 0.386 | −7.1% |
| EDTA-K2 sample-8 | 0.424 | 0.410 | −3.4% |
| EDTA-K2 sample-9 | 0.333 | 0.329 | −1.3% |
| EDTA-K2 sample-10 | 0.289 | 0.299 | 3.4% |
| | | Mean | −1.3% |
| | | variation | |

EDTA-K2-sample stability (2~8° C.)

| | | | |
|---|---|---|---|
| EDTA-K2 sample-11 | 0.630 | 0.613 | −2.6% |
| EDTA-K2 sample-12 | 0.427 | 0.421 | −1.3% |
| EDTA-K2 sample-13 | 0.984 | 0.997 | 1.3% |
| EDTA-K2 sample-14 | 0.305 | 0.284 | −6.7% |
| EDTA-K2 sample-15 | 0.632 | 0.614 | −2.8% |
| EDTA-K2 sample-16 | 0.166 | 0.168 | 1.0% |
| EDTA-K2 sample-17 | 0.328 | 0.318 | −3.0% |
| EDTA-K2 sample-18 | 0.192 | 0.193 | 0.2% |
| EDTA-K2 sample-19 | 0.508 | 0.465 | −8.5% |
| EDTA-K2 sample-20 | 0.392 | 0.361 | −8.1% |
| | | Mean | −3.0% |
| | | variation | |

8.7 The kits prepared using b-CTx_R_rec_134 and b-CTx_R_rec_145 were used to detect different samples according to the measurement procedures of 8.2. The results are shown in Table 13.

TABLE 13

| | | | |
|---|---|---|---|
| | 0 h | 12 h | |
| b-CTx_R_rec_134 and | concentration- | concentration- | |
| b-CTx_R_rec_145 | ng/mL | ng/mL | Variation |

Serum-sample stability (20~25° C.)

| | | | |
|---|---|---|---|
| Serum sample-1 | 0.692 | 0.631 | −8.7% |
| Serum sample-2 | 0.230 | 0.215 | −6.5% |
| Serum sample-3 | 0.399 | 0.383 | −4.0% |
| Serum sample-4 | 0.127 | 0.131 | 3.1% |
| Serum sample-5 | 0.684 | 0.620 | −9.3% |
| Serum sample-6 | 0.189 | 0.178 | −5.7% |
| Serum sample-7 | 0.474 | 0.478 | 1.0% |
| Serum sample-8 | 0.464 | 0.453 | −2.2% |
| Serum sample-9 | 0.369 | 0.356 | −3.4% |
| Serum sample-10 | 0.345 | 0.339 | −1.9% |
| | | Mean | −3.8% |
| | | variation | |

Serum-sample stability (2~8° C.)

| | | | |
|---|---|---|---|
| Serum sample-1 | 0.616 | 0.631 | 2.3% |
| Serum sample-2 | 0.483 | 0.493 | 2.0% |
| Serum sample-3 | 0.925 | 0.845 | −8.7% |
| Serum sample-4 | 0.324 | 0.328 | 1.1% |
| Serum sample-5 | 0.660 | 0.668 | 1.2% |
| Serum sample-6 | 0.146 | 0.144 | −1.7% |
| Serum sample-7 | 0.350 | 0.332 | −5.2% |
| Serum sample-8 | 0.213 | 0.207 | −3.0% |
| Serum sample-9 | 0.504 | 0.484 | −4.0% |
| Serum sample-10 | 0.388 | 0.367 | −5.5% |
| | | Mean | −2.1% |
| | | variation | |

Heparin lithium-sample stability (20~25° C.)

| | | | |
|---|---|---|---|
| Heparin lithium | 0.746 | 0.703 | −5.7% |
| sample-1 | | | |
| Heparin lithium | 0.284 | 0.270 | −5.0% |
| sample-2 | | | |
| Heparin lithium | 0.415 | 0.381 | −8.3% |
| sample-3 | | | |
| Heparin lithium | 0.134 | 0.128 | −4.3% |
| sample-4 | | | |
| Heparin lithium | 0.705 | 0.705 | 0.0% |
| sample-5 | | | |
| Heparin lithium | 0.182 | 0.166 | −8.5% |
| sample-6 | | | |
| Heparin lithium | 0.492 | 0.469 | −4.6% |
| sample-7 | | | |
| Heparin lithium | 0.478 | 0.450 | −6.0% |
| sample-8 | | | |
| Heparin lithium | 0.391 | 0.373 | −4.5% |
| sample-9 | | | |
| Heparin lithium | 0.357 | 0.336 | −5.9% |
| sample-10 | | | |
| | | Mean | −5.3% |
| | | variation | |

Heparin lithium-sample stability (2~8° C.)

| | | | |
|---|---|---|---|
| Heparin lithium | 0.689 | 0.660 | −4.2% |
| sample-11 | | | |
| Heparin lithium | 0.479 | 0.491 | 2.4% |
| sample-12 | | | |
| Heparin lithium | 0.942 | 0.884 | −6.2% |
| sample-13 | | | |
| Heparin lithium | 0.343 | 0.321 | −6.4% |
| sample-14 | | | |
| Heparin lithium | 0.706 | 0.678 | −3.9% |
| sample-15 | | | |
| Heparin lithium | 0.171 | 0.159 | −7.1% |
| sample-16 | | | |
| Heparin lithium | 0.384 | 0.378 | −1.7% |
| sample-17 | | | |
| Heparin lithium | 0.212 | 0.220 | 3.8% |
| sample-18 | | | |
| Heparin lithium | 0.571 | 0.569 | −0.4% |
| sample-19 | | | |
| Heparin lithium | 0.420 | 0.432 | 2.7% |
| sample-20 | | | |
| | | Mean | −2.1% |
| | | variation | |

EDTA-K2-sample stability (20~25° C.)

| | | | |
|---|---|---|---|
| EDTA-K2 sample-1 | 0.663 | 0.659 | −0.7% |
| EDTA-K2 sample-2 | 0.247 | 0.226 | −8.7% |
| EDTA-K2 sample-3 | 0.405 | 0.373 | −7.9% |
| EDTA-K2 sample-4 | 0.128 | 0.133 | 3.4% |
| EDTA-K2 sample-5 | 0.663 | 0.658 | −0.8% |
| EDTA-K2 sample-6 | 0.164 | 0.164 | −0.1% |
| EDTA-K2 sample-7 | 0.412 | 0.382 | −7.3% |
| EDTA-K2 sample-8 | 0.420 | 0.408 | −3.0% |
| EDTA-K2 sample-9 | 0.321 | 0.299 | −7.0% |
| EDTA-K2 sample-10 | 0.298 | 0.295 | −1.0% |
| | | Mean | −3.3% |
| | | variation | |

EDTA-K2-sample stability (2~8° C.)

| | | | |
|---|---|---|---|
| EDTA-K2 sample-11 | 0.575 | 0.550 | −4.3% |
| EDTA-K2 sample-12 | 0.439 | 0.463 | 5.6% |
| EDTA-K2 sample-13 | 0.930 | 0.881 | −5.2% |
| EDTA-K2 sample-14 | 0.313 | 0.300 | −4.0% |
| EDTA-K2 sample-15 | 0.637 | 0.635 | −0.4% |
| EDTA-K2 sample-16 | 0.156 | 0.145 | −6.9% |
| EDTA-K2 sample-17 | 0.302 | 0.281 | −6.7% |
| EDTA-K2 sample-18 | 0.191 | 0.197 | 3.4% |
| EDTA-K2 sample-19 | 0.512 | 0.476 | −6.9% |
| EDTA-K2 sample-20 | 0.361 | 0.347 | −4.0% |
| | | Mean | −3.0% |
| | | variation | |

8.8 The kits prepared using b-CTxM_hyb_015 and b-CTx_R_rec_137 were used to detect different samples according to the measurement procedures of 8.2. The results are shown in Table 14.

TABLE 14

| | | | |
|---|---|---|---|
| | 0 h | 12 h | |
| b-CTx_M_hyb_015 and | concentration- | concentration- | |
| b-CTx_R_rec_137 | ng/mL | ng/mL | Variation |

Serum-sample stability (20~25° C.)

| | | | |
|---|---|---|---|
| Serum sample-1 | 0.692 | 0.631 | −8.8% |
| Serum sample-2 | 0.222 | 0.215 | −3.0% |
| Serum sample-3 | 0.370 | 0.386 | 4.2% |
| Serum sample-4 | 0.126 | 0.127 | 1.1% |
| Serum sample-5 | 0.640 | 0.610 | −4.8% |
| Serum sample-6 | 0.173 | 0.166 | −4.0% |
| Serum sample-7 | 0.474 | 0.453 | −4.2% |
| Serum sample-8 | 0.438 | 0.416 | −5.0% |
| Serum sample-9 | 0.350 | 0.324 | −7.6% |
| Serum sample-10 | 0.326 | 0.315 | −3.2% |
| | | Mean | −3.5% |
| | | variation | |

Serum-sample stability (2~8° C.)

| | | | |
|---|---|---|---|
| Serum sample-1 | 0.561 | 0.518 | −7.6% |
| Serum sample-2 | 0.455 | 0.470 | 3.2% |
| Serum sample-3 | 0.925 | 0.860 | −7.1% |
| Serum sample-4 | 0.304 | 0.313 | 2.7% |
| Serum sample-5 | 0.616 | 0.582 | −5.5% |
| Serum sample-6 | 0.140 | 0.130 | −6.8% |
| Serum sample-7 | 0.323 | 0.298 | −7.8% |
| Serum sample-8 | 0.208 | 0.204 | −2.2% |
| Serum sample-9 | 0.437 | 0.399 | −8.6% |
| Serum sample-10 | 0.361 | 0.356 | −1.5% |
| | | Mean | −4.1% |
| | | variation | |

Heparin lithium-sample stability (20~25° C.)

| | | | |
|---|---|---|---|
| Heparin lithium | 0.706 | 0.641 | −9.2% |
| sample-1 | | | |
| Heparin lithium | 0.263 | 0.249 | −5.4% |
| sample-2 | | | |
| Heparin lithium | 0.399 | 0.379 | −5.1% |
| sample-3 | | | |
| Heparin lithium | 0.135 | 0.123 | −9.3% |
| sample-4 | | | |
| Heparin lithium | 0.690 | 0.650 | −5.9% |
| sample-5 | | | |
| Heparin lithium | 0.171 | 0.156 | −9.0% |
| sample-6 | | | |
| Heparin lithium | 0.466 | 0.481 | 3.2% |
| sample-7 | | | |
| Heparin lithium | 0.436 | 0.412 | −5.6% |
| sample-8 | | | |
| Heparin lithium | 0.363 | 0.340 | −6.4% |
| sample-9 | | | |
| Heparin lithium | 0.330 | 0.324 | −1.6% |
| sample-10 | | | |
| | | Mean | −5.4% |
| | | variation | |

Heparin lithium-sample stability (2~8° C.)

| | | | |
|---|---|---|---|
| Heparin lithium | 0.648 | 0.698 | 7.7% |
| sample-11 | | | |
| Heparin lithium | 0.460 | 0.429 | −6.7% |
| sample-12 | | | |
| Heparin lithium | 0.947 | 0.891 | −5.8% |
| sample-13 | | | |
| Heparin lithium | 0.355 | 0.380 | 6.8% |
| sample-14 | | | |
| Heparin lithium | 0.721 | 0.669 | −7.1% |
| sample-15 | | | |
| Heparin lithium | 0.174 | 0.161 | −7.7% |
| sample-16 | | | |
| Heparin lithium | 0.393 | 0.390 | −0.8% |
| sample-17 | | | |
| Heparin lithium | 0.197 | 0.180 | −8.8% |
| sample-18 | | | |
| Heparin lithium | 0.571 | 0.550 | −3.7% |
| sample-19 | | | |
| Heparin lithium | 0.410 | 0.389 | −5.2% |
| sample-20 | | | |
| | | Mean | −3.1% |
| | | variation | |

EDTA-K2-sample stability (20~25° C.)

| | | | |
|---|---|---|---|
| EDTA-K2 sample-1 | 0.692 | 0.653 | −5.7% |
| EDTA-K2 sample-2 | 0.246 | 0.228 | −7.5% |
| EDTA-K2 sample-3 | 0.411 | 0.385 | −6.4% |
| EDTA-K2 sample-4 | 0.135 | 0.135 | 0.1% |
| EDTA-K2 sample-5 | 0.644 | 0.664 | 3.1% |
| EDTA-K2 sample-6 | 0.156 | 0.144 | −7.4% |
| EDTA-K2 sample-7 | 0.415 | 0.395 | −4.8% |
| EDTA-K2 sample-8 | 0.423 | 0.412 | −2.5% |
| EDTA-K2 sample-9 | 0.332 | 0.331 | −0.4% |
| EDTA-K2 sample-10 | 0.289 | 0.269 | −7.1% |
| | | Mean | −3.9% |
| | | variation | |

EDTA-K2-sample stability (2~8° C.)

| | | | |
|---|---|---|---|
| EDTA-K2 sample-11 | 0.604 | 0.584 | −3.2% |
| EDTA-K2 sample-12 | 0.428 | 0.426 | −0.4% |
| EDTA-K2 sample-13 | 0.951 | 0.921 | −3.2% |
| EDTA-K2 sample-14 | 0.314 | 0.295 | −6.1% |
| EDTA-K2 sample-15 | 0.638 | 0.604 | −5.4% |
| EDTA-K2 sample-16 | 0.161 | 0.151 | −5.9% |
| EDTA-K2 sample-17 | 0.320 | 0.342 | 6.9% |
| EDTA-K2 sample-18 | 0.194 | 0.196 | 1.2% |
| EDTA-K2 sample-19 | 0.512 | 0.525 | 2.5% |
| EDTA-K2 sample-20 | 0.383 | 0.364 | −5.0% |
| | | Mean | −1.9% |
| | | variation | |

At present, the requirements of β-CTx immunodiagnostic kits commercially available from international manufacturers for sample stability are as follows:
1. Serum: samples should be used within 6 h if stored at 20˜25° C., and samples should be used within 8 h if stored at 2˜8° C.;
2. Heparin lithium plasma: samples should be used within 4 h if stored at 20˜25° C., and samples should be used within 8 h if stored at 2˜8° C.;
3. EDTA-K2 plasma: samples should be used within 24 h if stored at 20˜25° C., and samples should be used within 8 days if stored at 2˜8° C.

It can be seen from Table 9-Table 14 that compared with the β-CTx immunodiagnostic kits commercially available from international manufacturers, the kits prepared by the antibodies of the present application reduce the requirements for storage time of samples, with the following details:
1. Serum: the storage time of samples can be extended to 12 h at 20˜25° C., and extended to 24 h at 2˜8° C.;
2. Heparin lithium plasma: the storage time of samples can be extended to 12 h at 20˜25° C., and extended to 24 h at 2˜8° C.;
3. EDTA-K2 plasma: samples of 24 h can be used if stored at 20˜25° C. and samples of 8 days can be used if stored at 2˜8° C.

### Example 9 Assessment on Consistency Between Serum, Heparin Lithium Plasma and EDTA-K2 Plasma Samples

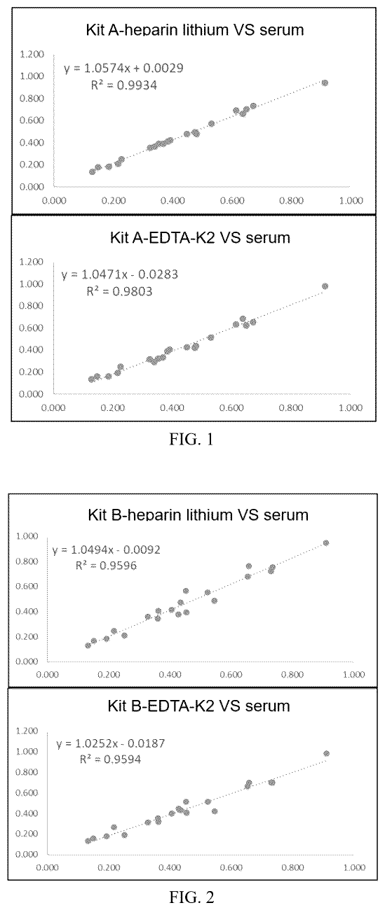

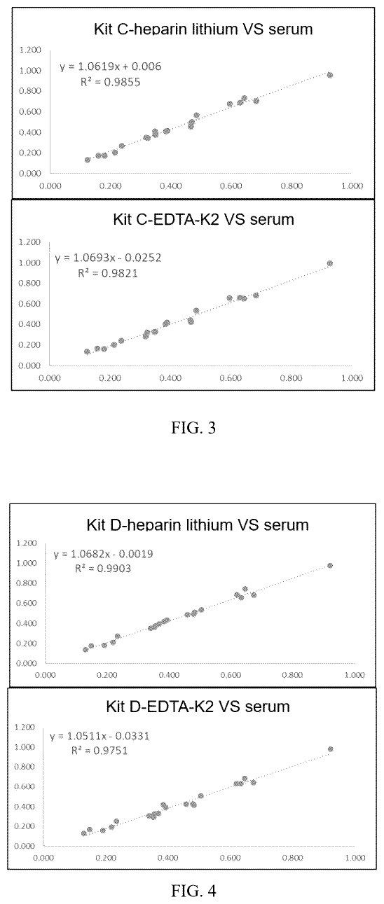

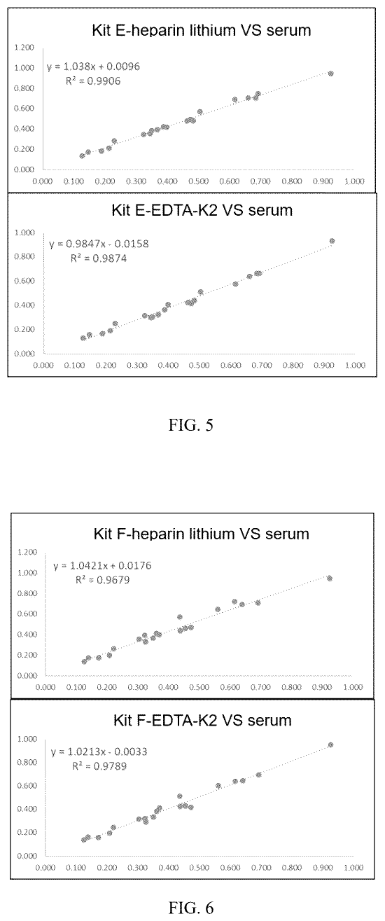

Next, the kits in Example 8 were assessed for the consistency between the results of for different sample types, and the results are shown in FIGS. 1-6.

It can be seen from FIGS. 1-6 that the kits prepared using antibodies of the present application have high consistency when testing different types of samples.

### Example 10 Assessment on Specificity

In addition to the previously verified α-CTx, the anti-interference ability of the antibodies of the present application was further verified. In this experiment, common endogenous interfering substances including N-terminal midfragment of osteocalcin (N-MID OC), Type 1 procollagen N-terminal propeptide (P1NP) and parathyroid hormone (PTH) were tested, and serum samples containing the above interfering substances were prepared. The test procedures were performed as described in Example 8.

The cross-reactivity percentage was calculated from the measured concentration/supplemented concentration.

The test results are shown in Table 15.

TABLE 15

| Cross-reactivity (%) | N-MID OC | PINP | PTH |
|---|---|---|---|
| Kit A | 0.000% | 0.001% | 0.000% |
| Kit B | 0.003% | 0.001% | 0.000% |
| Kit C | 0.012% | 0.000% | 0.000% |
| Kit D | 0.009% | 0.000% | 0.000% |
| Kit E | 0.005% | 0.001% | 0.000% |
| Kit F | 0.001% | 0.000% | 0.000% |

It can be seen from Table 15 that the kits of the present application have low cross-reactivity with common β-CTx analogues and have good specificity.

### Example 11 Assessment on Detection Range and Linearity

A quantitative determination method that established a linear range was used, and 11 samples with known concentration levels were selected within the expected determination range. The sample with the highest concentration close to the upper limit of the linear range was diluted to several concentrations at a certain ratio, and the sample with the lowest concentration was close to the lower limit of the linear range. The measurement results and the theoretical results were linearly fitted, and the correlation coefficient R² was calculated in the linear range.

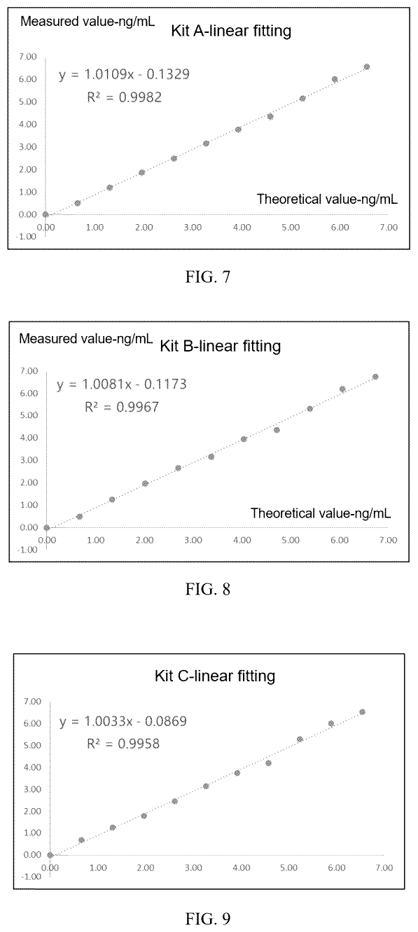

The results show that: the determination ranges of the kits of the present application all can reach 6.00 ng/mL or above, and the linear R² is greater than 0.99 between 0 ng/mL and 6.00 ng/mL, meeting the performance requirements of the kits. FIGS. 7-9 show exemplary fitting results.

### Example 12 Assessment on Clinical Diagnosis Effectiveness

The gold standard for the diagnosis of osteoporosis is bone mineral density. However, by the measurement and analysis of bone turnover markers, the type of osteoporosis can be further clarified, which plays a crucial role in guiding clinicians' treatment decisions.

Osteoporosis can be classified into high-turnover osteoporosis and low-turnover osteoporosis according to the bone turnover rate. Generally, for patients with high-turnover osteoporosis, anti-bone resorption therapeutic regimens are usually given clinically. For patients with low-turnover osteoporosis, basic calcium agents or therapeutic regimens that promote bone formation will be given clinically.

### Patient 1:

female, 63 years old, with GRF (glomerular filtration rate) of 93.6 ml/min that is normal, blood calcium level of 2.19 mmol/L that is normal, blood phosphorus level of 1.31 mmol/L that is normal, PTH (parathyroid hormone) level of 72.29 μg/ml that is normal, VD-T (total 25-OH vitamin D) of 23.26 ng/ml that is insufficient, TP1NP (total Type 1 procollagen N-terminal propeptide) of 86.71 ng/ml that is of high turnover type, and the patient's clinical manifestation was waist and back pain for 6 months.

Based on the above information, the clinical diagnosis result was primary high-turnover osteoporosis.

According to the relevant clinical guidelines for osteoporosis, the β-CTx level exceeding 1.0 ng/ml indicates high-turnover osteoporosis. β-CTx test was performed using kit C, the test result was 1.317 ng/ml, confirming diagnosis of high-turnover osteoporosis. This indicates that the diagnostic conclusion based on the test results of the β-CTx kit of the present application is consistent with the clinical diagnosis result.

### Patient 2:

female, 62 years old, with GRF (glomerular filtration rate) of 100.53 ml/min that is normal, blood calcium level of 2.28 mmol/L that is normal, blood phosphorus level of 1.26 mmol/L that is normal, PTH (parathyroid hormone) level of 40.97 μg/ml that is normal, VD-T (total 25-OH vitamin D) of 21.25 ng/ml that is insufficient, TP1NP (total Type 1 procollagen N-terminal propeptide) of 123.35 ng/ml that is of high turnover type, and the patient's clinical manifestation was waist and back pain for 8 months.

Based on the above information, the clinical diagnosis result was primary high-turnover osteoporosis.

β-CTx test was performed using kit C, and the test result was 1.256 ng/ml, confirming diagnosis of high-turnover osteoporosis according to the relevant clinical guidelines. This indicates that the diagnostic conclusion based on the test results of the β-CTx kit of the present application is consistent with the clinical diagnosis result.

### Patient 3:

male, 78 years old, with GRF (glomerular filtration rate) of 101.98 ml/min that is normal, blood calcium level of 2.17 mmol/L that is normal, blood phosphorus level of 1.01 mmol/L that is normal, PTH (parathyroid hormone) level of 67.85 μg/ml that is normal, VD-T (total 25-OH vitamin D) of 34.66 ng/ml that is normal, TP1NP (total Type 1 procollagen N-terminal propeptide) of 16.48 ng/ml that is of low turnover type, and the patient's clinical manifestation was waist and back pain for 9 months.

Based on the above information, the clinical diagnosis result was primary low-turnover osteoporosis.

β-CTx test was performed using kit C, and the test result was 0.043 ng/ml, confirming diagnosis of low-turnover osteoporosis according to the relevant clinical guidelines. This indicates that the diagnostic conclusion based on the test results of the β-CTx kit of the present application is consistent with the clinical diagnosis result.

This example shows that the diagnosis results of osteoporosis based on the test results of the β-CTx kits of the present application are consistent with the clinical diagnosis results obtained by comprehensive evaluation of multiple biochemical indicators and the clinical manifestations of patients.

1. An antibody or an antigen-binding fragment thereof capable of binding to 3-CTx, comprising:
a heavy chain variable region (VH) and a light chain variable region (VL),
wherein the heavy chain variable region comprises HCDR1 having an amino acid sequence as set forth in any one of SEQ ID NO: 1, 7, 13, 19, 25, 31, 37, 43, 49 or 55 or a variant thereof, HCDR2 having an amino acid sequence as set forth in any one of SEQ ID NO: 2, 8, 14, 20, 26, 32, 38, 44, 50 or 56 or a variant thereof, and HCDR3 having an amino acid sequence as set forth in any one of SEQ ID NO: 3, 9, 15, 21, 27, 33, 39, 45, 51 or 57 or a variant thereof,
wherein, the light chain variable region comprises LCDR1 having an amino acid sequence as set forth in any one of SEQ ID NO: 4, 10, 16, 22, 28, 34, 40, 46, 52 or 58 or a variant thereof, LCDR2 having an amino acid sequence as set forth in any one of SEQ ID NO: 5, 11, 17, 23, 29, 35, 41, 47, 53 or 59 or a variant thereof, and LCDR3 having an amino acid sequence as set forth in any one of SEQ ID NO: 6, 12, 18, 24, 30, 36, 42, 48, 54 or 60 or a variant thereof, and
wherein each variant comprises one or more amino acid mutations compared to the amino acid sequence from which it is derived, and each amino acid mutation is selected from amino acid substitution, deletion or addition; preferably, the substitution is a conservative substitution; preferably, each variant has at least 80%, at least 90%, or at least 95% identity to the amino acid sequence from which it is derived.

2. The antibody or the antigen-binding fragment thereof according to claim 1, wherein:
HCDR1, HCDR2, HCDR3, LCDR1, LCDR2 and LCDR3 include sequences that have at least 80%, at least 90%, or at least 95% identity to SEQ ID NOs: 1-6, respectively;
HCDR1, HCDR2, HCDR3, LCDR1, LCDR2 and LCDR3 include sequences that have at least 80%, at least 90%, or at least 95% identity to SEQ ID NOs: 7-12, respectively;
HCDR1, HCDR2, HCDR3, LCDR1, LCDR2 and LCDR3 include sequences that have at least 80%, at least 90%, or at least 95% identity to SEQ ID NOs: 13-18, respectively;
HCDR1, HCDR2, HCDR3, LCDR1, LCDR2 and LCDR3 include sequences that have at least 80%, at least 90%, or at least 95% identity to SEQ ID NOs: 19-24, respectively;
HCDR1, HCDR2, HCDR3, LCDR1, LCDR2 and LCDR3 include sequences that have at least 80%, at least 90%, or at least 95% identity to SEQ ID NOs: 25-30, respectively;
HCDR1, HCDR2, HCDR3, LCDR1, LCDR2 and LCDR3 include sequences that have at least 80%, at least 90%, or at least 95% identity to SEQ ID NOs: 31-36, respectively;
HCDR1, HCDR2, HCDR3, LCDR1, LCDR2 and LCDR3 include sequences that have at least 80%, at least 90%, or at least 95% identity to SEQ ID NOs: 37-42, respectively;
HCDR1, HCDR2, HCDR3, LCDR1, LCDR2 and LCDR3 include sequences that have at least 80%, at least 90%, or at least 95% identity to SEQ ID NOs: 43-48, respectively;
HCDR1, HCDR2, HCDR3, LCDR1, LCDR2 and LCDR3 include sequences that have at least 80%, at least 90%, or at least 95% identity to SEQ ID NOs: 49-54, respectively; or
HCDR1, HCDR2, HCDR3, LCDR1, LCDR2 and LCDR3 include sequences that have at least 80%, at least 90%, or at least 95% identity to SEQ ID NOs: 55-60, respectively.

3. The antibody or the antigen-binding fragment thereof according to claim 1, wherein:
HCDR1, HCDR2, HCDR3, LCDR1, LCDR2 and LCDR3 include sequences as set forth in SEQ ID NOs: 1-6, respectively;
HCDR1, HCDR2, HCDR3, LCDR1, LCDR2 and LCDR3 include sequences as set forth in SEQ ID NOs: 7-12, respectively;
HCDR1, HCDR2, HCDR3, LCDR1, LCDR2 and LCDR3 include sequences as set forth in SEQ ID NOs: 13-18, respectively;
HCDR1, HCDR2, HCDR3, LCDR1, LCDR2 and LCDR3 include sequences as set forth in SEQ ID NOs: 19-24, respectively;
HCDR1, HCDR2, HCDR3, LCDR1, LCDR2 and LCDR3 include sequences as set forth in SEQ ID NOs: 25-30, respectively;
HCDR1, HCDR2, HCDR3, LCDR1, LCDR2 and LCDR3 include sequences as set forth in SEQ ID NOs: 31-36, respectively;
HCDR1, HCDR2, HCDR3, LCDR1, LCDR2 and LCDR3 include sequences as set forth in SEQ ID NOs: 37-42, respectively;
HCDR1, HCDR2, HCDR3, LCDR1, LCDR2 and LCDR3 include sequences as set forth in SEQ ID NOs: 43-48, respectively;
HCDR1, HCDR2, HCDR3, LCDR1, LCDR2 and LCDR3 include sequences as set forth in SEQ ID NOs: 49-54, respectively; or
HCDR1, HCDR2, HCDR3, LCDR1, LCDR2 and LCDR3 include sequences as set forth in SEQ ID NOs: 55-60, respectively.

4. The antibody or the antigen-binding fragment thereof according to claim 1, wherein an antigen-binding epitope of the antibody or the antigen-binding fragment thereof is selected from one or more of the group consisting of: AH(betaD)G, AH(betaD)GG, AH(betaD)GGR and AH(betaD)GGR-COOH.

5. An antibody or an antigen-binding fragment thereof, wherein the antibody is capable of being produced by hybridoma H-215 deposited with VKPM, or being produced by hybridoma H-216 deposited with VKPM, or being produced by hybridoma H-217 deposited with VKPM.

6. An antibody or an antigen-binding fragment thereof, wherein the antibody:
is produced based on a plasmid deposited with VKPM under a deposit number of B-14772 and a plasmid deposited with VKPM under a deposit number of B-14773;
is produced based on a plasmid deposited with VKPM under a deposit number of B-14774 and a plasmid deposited with VKPM under a deposit number of B-14775;
is produced based on a plasmid deposited with VKPM under a deposit number of B-14776 and a plasmid deposited with VKPM under a deposit number of B-14777;
is produced based on a plasmid deposited with VKPM under a deposit number of B-14778 and a plasmid deposited with VKPM under a deposit number of B-14779;
is produced based on a plasmid deposited with VKPM under a deposit number of B-14780 and a plasmid deposited with VKPM under a deposit number of B-14781;
is produced based on a plasmid deposited with VKPM under a deposit number of B-14782 and a plasmid deposited with VKPM under a deposit number of B-14783; or
is produced based on a plasmid deposited with VKPM under a deposit number of B-14784 and a plasmid deposited with VKPM under a deposit number of B-14785.

7. An isolated nucleic acid molecule encoding the antibody or the antigen-binding fragment thereof according to claim 1.

8. An expression vector comprising the isolated nucleic acid molecule according to claim 7.

9. A host cell comprising the isolated nucleic acid molecule according to claim 7.

10. A kit, wherein the kit is for detecting β-CTx in a sample and the kit comprises a first antibody or an antigen-binding fragment thereof and a second antibody or an antigen-binding fragment thereof, wherein the first antibody or the antigen-binding fragment thereof or the second antibody or the antigen-binding fragment thereof is selected from the antibody or the antigen-binding fragment thereof of claim 1.

11. The kit according to claim 10, wherein the sample is serum or plasma, such as heparin plasma or EDTA plasma, preferably serum or plasma collected after 8 h, 12 h, 24 h, 2 days, 4 days, or 8 days.

12. The kit according to claim 10, wherein the first antibody is different from the second antibody, and the first antibody and the second antibody each independently comprises:
HCDR1, HCDR2, HCDR3, LCDR1, LCDR2 and LCDR3 including sequences that have at least 80%, at least 90%, at least 95%, or 100% identity to SEQ ID NOs: 1-6, respectively;
HCDR1, HCDR2, HCDR3, LCDR1, LCDR2 and LCDR3 including sequences that have at least 80%, at least 90%, at least 95%, or 100% identity to SEQ ID NOs: 7-12, respectively;
HCDR1, HCDR2, HCDR3, LCDR1, LCDR2 and LCDR3 including sequences that have at least 80%, at least 90%, at least 95%, or 100% identity to SEQ ID NOs: 13-18, respectively;
HCDR1, HCDR2, HCDR3, LCDR1, LCDR2 and LCDR3 including sequences that have at least 80%, at least 90%, at least 95%, or 100% identity to SEQ ID NOs: 19-24, respectively;
HCDR1, HCDR2, HCDR3, LCDR1, LCDR2 and LCDR3 including sequences that have at least 80%, at least 90%, at least 95%, or 100% identity to SEQ ID NOs: 25-30, respectively;
HCDR1, HCDR2, HCDR3, LCDR1, LCDR2 and LCDR3 including sequences that have at least 80%, at least 90%, at least 95%, or 100% identity to SEQ ID NOs: 31-36, respectively;
HCDR1, HCDR2, HCDR3, LCDR1, LCDR2 and LCDR3 including sequences that have at least 80%, at least 90%, at least 95%, or 100% identity to SEQ ID NOs: 37-42, respectively;
HCDR1, HCDR2, HCDR3, LCDR1, LCDR2 and LCDR3 including sequences that have at least 80%, at least 90%, at least 95%, or 100% identity to SEQ ID NOs: 43-48, respectively;
HCDR1, HCDR2, HCDR3, LCDR1, LCDR2 and LCDR3 including sequences that have at least 80%, at least 90%, at least 95%, or 100% identity to SEQ ID NOs: 49-54, respectively; or
HCDR1, HCDR2, HCDR3, LCDR1, LCDR2 and LCDR3 including sequences that have at least 80%, at least 90%, at least 95%, or 100% identity to SEQ ID NOs: 55-60, respectively.

13. The kit according to claim 12, wherein
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 55-60, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 1-6, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 25-30, respectively;
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 37-42, respectively;
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 13-18, respectively;
the first antibody comprises HCDR1, HCDR2, HCDR3, LCDR1, LCDR2 and LCDR3 including sequences as set forth in SEQ ID NOs: 13-18, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 13-18, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 19-24, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 19-24, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 25-30, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 25-30, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 31-36, respectively; and the second antibody comprises HCDR1, HCDR2, HCDR3, LCDR1, LCDR2 and LCDR3 including sequences as set forth in SEQ ID NOs: 13-18, respectively;
the first antibody comprises HCDR1, HCDR2, HCDR3, LCDR1, LCDR2 and LCDR3 including sequences as set forth in SEQ ID NOs: 31-36, respectively; and the second antibody comprises HCDR1, HCDR2, HCDR3, LCDR1, LCDR2 and LCDR3 including sequences as set forth in SEQ ID NOs: 25-30, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 13-18, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 49-54, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 19-24, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 25-30, respectively; or
the first antibody comprises HCDR1, HCDR2, HCDR3, LCDR1, LCDR2 and LCDR3 including sequences as set forth in SEQ ID NOs: 55-60, respectively; and the second antibody comprises HCDR1, HCDR2, HCDR3, LCDR1, LCDR2 and LCDR3 including sequences as set forth in SEQ ID NOs: 25-30, respectively.

14. The kit according to claim 10, wherein a cross-reactivity percentage of the kit with α-Ctx is not higher than 0.20%, such as not higher than 0.10%, 0.05%, or 0.02%.

15. The kit according to claim 13, wherein
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 55-60, respectively; and the second antibody comprises HCDR1, HCDR2, HCDR3, LCDR1, LCDR2 and LCDR3 including sequences as set forth in SEQ ID NOs: 1-6, respectively; or
the first antibody comprises HCDR1, HCDR2, HCDR3, LCDR1, LCDR2 and LCDR3 including sequences as set forth in SEQ ID NOs: 1-6, respectively; and the second antibody comprises HCDR1, HCDR2, HCDR3, LCDR1, LCDR2 and LCDR3 including sequences as set forth in SEQ ID NOs: 7-12, respectively.

16. The kit according to claim 10, wherein
the first antibody comprises HCDR1, HCDR2, HCDR3, LCDR1, LCDR2 and LCDR3 including sequences as set forth in SEQ ID NOs: 13-18, respectively; and the second antibody comprises HCDR1, HCDR2, HCDR3, LCDR1, LCDR2 and LCDR3 including sequences as set forth in SEQ ID NOs: 37-42, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 31-36, respectively;
the first antibody comprises HCDR1, HCDR2, HCDR3, LCDR1, LCDR2 and LCDR3 including sequences as set forth in SEQ ID NOs: 1-6, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 55-60, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 19-24, respectively; and the second antibody comprises HCDR1, HCDR2, HCDR3, LCDR1, LCDR2 and LCDR3 including sequences as set forth in SEQ ID NOs: 1-6, respectively; or
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 25-30, respectively.

17. The kit according to claim 10, wherein the first antibody is a capture antibody, and the second antibody is a detection antibody.

18. A method for diagnosing osteoporosis in a subject, comprising:
using a first antibody or an antigen-binding fragment thereof and a second antibody or an antigen-binding fragment thereof to detect a sample from the subject;
determining an amount of β-CTx in the sample; and
determining whether the subject has osteoporosis based on the determined amount of β-CTx,
wherein one or both of the first antibody or the antigen-binding fragment thereof and the second antibody or the antigen-binding fragment thereof is selected from the antibody or the antigen-binding fragment thereof of claim 1.

19. The method according to claim 18, wherein the first antibody is different from the second antibody, and the first antibody and the second antibody each independently comprises:
HCDR1, HCDR2, HCDR3, LCDR1, LCDR2 and LCDR3 including sequences that have at least 80%, at least 90%, at least 95%, or 100% identity to SEQ ID NOs: 1-6, respectively;
HCDR1, HCDR2, HCDR3, LCDR1, LCDR2 and LCDR3 including sequences that have at least 80%, at least 90%, at least 95%, or 100% identity to SEQ ID NOs: 7-12, respectively;
HCDR1, HCDR2, HCDR3, LCDR1, LCDR2 and LCDR3 including sequences that have at least 80%, at least 90%, at least 95%, or 100% identity to SEQ ID NOs: 13-18, respectively;
HCDR1, HCDR2, HCDR3, LCDR1, LCDR2 and LCDR3 including sequences that have at least 80%, at least 90%, at least 95%, or 100% identity to SEQ ID NOs: 19-24, respectively;
HCDR1, HCDR2, HCDR3, LCDR1, LCDR2 and LCDR3 including sequences that have at least 80%, at least 90%, at least 95%, or 100% identity to SEQ ID NOs: 25-30, respectively;
HCDR1, HCDR2, HCDR3, LCDR1, LCDR2 and LCDR3 including sequences that have at least 80%, at least 90%, at least 95%, or 100% identity to SEQ ID NOs: 31-36, respectively;
HCDR1, HCDR2, HCDR3, LCDR1, LCDR2 and LCDR3 including sequences that have at least 80%, at least 90%, at least 95%, or 100% identity to SEQ ID NOs: 37-42, respectively;
HCDR1, HCDR2, HCDR3, LCDR1, LCDR2 and LCDR3 including sequences that have at least 80%, at least 90%, at least 95%, or 100% identity to SEQ ID NOs: 43-48, respectively;
HCDR1, HCDR2, HCDR3, LCDR1, LCDR2 and LCDR3 including sequences that have at least 80%, at least 90%, at least 95%, or 100% identity to SEQ ID NOs: 49-54, respectively; or
HCDR1, HCDR2, HCDR3, LCDR1, LCDR2 and LCDR3 including sequences that have at least 80%, at least 90%, at least 95%, or 100% identity to SEQ ID NOs: 55-60, respectively.

20. The method according to claim 18, wherein the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 55-60, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 1-6, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 25-30, respectively;
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 37-42, respectively;
the first antibody comprises HCDR1, HCDR2, HCDR3, LCDR1, LCDR2 and LCDR3 including sequences as set forth in SEQ ID NOs: 43-48, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 13-18, respectively;
the first antibody comprises HCDR1, HCDR2, HCDR3, LCDR1, LCDR2 and LCDR3 including sequences as set forth in SEQ ID NOs: 13-18, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 13-18, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 19-24, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 19-24, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 25-30, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 25-30, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 7-12, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 1-6, respectively;
the first antibody comprises HCDR1, HCDR2, HCDR3, LCDR1, LCDR2 and LCDR3 including sequences as set forth in SEQ ID NOs: 31-36, respectively; and the second antibody comprises HCDR1, HCDR2, HCDR3, LCDR1, LCDR2 and LCDR3 including sequences as set forth in SEQ ID NOs: 13-18, respectively;
the first antibody comprises HCDR1, HCDR2, HCDR3, LCDR1, LCDR2 and LCDR3 including sequences as set forth in SEQ ID NOs: 31-36, respectively; and the second antibody comprises HCDR1, HCDR2, HCDR3, LCDR1, LCDR2 and LCDR3 including sequences as set forth in SEQ ID NOs: 25-30, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 13-18, respectively;
the first antibody comprises HCDR1, HCDR2, HCDR3, LCDR1, LCDR2 and LCDR3 including sequences as set forth in SEQ ID NOs: 37-42, respectively; and the second antibody comprises HCDR1, HCDR2, HCDR3, LCDR1, LCDR2 and LCDR3 including sequences as set forth in SEQ ID NOs: 49-54, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 19-24, respectively;
the first antibody comprises HCDR1, HCDR2, HCDR3, LCDR1, LCDR2 and LCDR3 including sequences as set forth in SEQ ID NOs: 49-54, respectively; and the second antibody comprises HCDR1, HCDR2, HCDR3, LCDR1, LCDR2 and LCDR3 including sequences as set forth in SEQ ID NOs: 25-30, respectively; or
the first antibody comprises HCDR1, HCDR2, HCDR3, LCDR1, LCDR2 and LCDR3 including sequences as set forth in SEQ ID NOs: 55-60, respectively; and the second antibody comprises HCDR1, HCDR2, HCDR3, LCDR1, LCDR2 and LCDR3 including sequences as set forth in SEQ ID NOs: 25-30, respectively.
